(19) United States
(12) Patent Application Publication
(10) Pub. No.: US 2009/0022759 A1
(43) Pub. Date: Jan. 22, 2009

(54) ADENOVIRUS VECTOR AND METHOD TO MANIPULATE THE ADENOVIRUS GENOME

(76) Inventors: Hans Gerhard Burgert, Coventry (GB); Ruzsics Zsolt, Munich (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/572,237

(22) PCT Filed: Jul. 13, 2005
(86) PCT No.: PCT/GB05/02757

§ 371 Date: Feb. 28, 2008

(30) Foreign Application Priority Data
Jul. 20, 2004 (GB) 0416161.8
Jul. 20, 2004 (GB) 0416163.4
May 20, 2005 (GB) 0510293.4

(51) Int. Cl. A61K 39/00; C12N 7/00; C12N 5/06; A61P 31/00; A61K 35/00; A61K 35/12

(52) U.S. Cl. 424/199.1; 435/235.1; 435/325; 424/93.2; 424/93.21

(57) ABSTRACT

Adenoviruses (Ads) and vectors derived thereof have been used for somatic gene therapy, gene therapy of cancer and gene therapy of infectious diseases/vaccination. To date, almost all trials are based on the well established Ad5-based vectors. Pre-existing immunity and the limited targeting specificity of Ad5 makes it desirable to exploit new Ad serotypes for these therapeutic avenues. This is hampered by the limited number of cloned Ad genomes and the difficulty to manipulate them genetically. We describe an isolated adenovirus, and/or a variant adenovirus that is optionally modified to include a heterologous nucleic acid molecule and pharmaceutical compositions comprising said adenovirus. This adenovirus has a lower pre-existing immunity and exhibits interesting targeting activities for a variety of tissues and cells, and may be particularly useful for transduction of dendritic cells and other leukocytes and or leukocyte based tumours. We also describe new methods to clone and manipulate adenoviral genomes.

The invention relates to an isolated adenovirus, and/or a variant adenovirus; optionally said adenovirus is modified to include a heterologous nucleic acid molecule; pharmaceutical compositions comprising said adenovirus; and methods to construct and manipulate any recombinant adenovirus genome.

Adenoviruses (Ads) were first isolated in 1953 by Rowe et al. (Rowe et al., 1953) who were trying to establish cell-lines from adenoidal tissue of children removed during tonsillectomy and from military recruits with febrile illness. Adenoviruses are widespread in nature, infecting birds, mammals and man. Belonging to the family Adenoviridae and the genus Mastadenovirus, over 50 human adenovirus serotypes have been classified within 6 subgenera (A-F), according to their hemaggultination pattern, their DNA homology and other criteria (Shenk, 2001). The most prevalent serotypes are those of subgenus C (1, 2, 5 and 6). Together with some serotypes of subgenus B and E these viruses are a frequent cause of acute upper respiratory tract (URT) infections, i.e. “colds”, and other respiratory pathologies. They have been shown to cause some 5% of acute respiratory diseases in children under 5 years of age and 10% of the pneumonias (Horwitz, 2001). In addition, Ads also cause a number of other types of infection often associated with the eye (e.g conjunctivitis and epidemic keratoconjunctivitis), the gastrointestinal tract (e.g. gastroenteritis) or the urogenital tract (e.g cystitis). The organ tropism is distinct for different human adenovirus subgenera. Often these diseases are self-limiting, unless the immune system is suppressed, such as in transplantation patients (Horwitz, 2001). Ads have also been used therapeutically for vaccination and for gene therapy (Horwitz, 2001; Russell, 2000).

Gene therapy aims at treating both genetic (e.g. cancer, haemophilia) and infectious diseases (e.g. AIDS) by introducing new genetic material into selected cells. The major challenge is to deliver the gene safely and efficiently into the desired target cells. Among the biggest physical hurdles that gene delivery technologies have to overcome are the various lipid membranes of the cell, e.g. the plasma membrane or the nuclear membrane. The plasma membrane is impermeable to charged macromolecules such as DNA and RNA. Numerous different gene delivery methods using chemical, physical and biological principles are known. Virus-mediated transduction, liposome-based and receptor mediated transfection reagents are the most widely used techniques for the introduction of the desired gene into target cells. One of the most successful examples for virus-mediated transfer of a gene of interest into appropriate cell lines or tissues is through the use of recombinant adenoviruses. The gene of interest is introduced into a modified adenovirus genome (vector) and amplified in vitro. Subsequently, the recombinant adenovirus particles are used to transduce the target cells (Imperiale and Kochanek, 2004).

In order to use adenovirus-based vectors for gene therapy, the virus has to be modified. To eliminate or minimise its disease-causing potential the virus is usually rendered replication-deficient or conditionally replicative. In addition, room within the Ad genome has to be created for the therapeutic gene of interest. This is achieved by eliminating parts of the viral genome that are either not essential or that can be complemented in certain cell lines. Adenovirus gene expression is a two-phase process that can be divided into an early and late stage, occurring before and after the onset of viral DNA replication, respectively (Shenk, 2001). The early regions are E1, E2, E3 and E4. The E1 gene products are further subdivided into E1A and E1B. E1 gene products are essential for efficient replication of the virus. Conventionally, the modifications of the Ad genome involve the deletion of the E1 and part of the “nonessential” E3 region (first generation vector). Other regions also have been deleted. In the extreme case, so-called gutless or high capacity vectors have been developed which lack essentially all Ad-coding sequences (Volpers and Kochanek, 2004). In animal models, these types of vectors seem to exhibit a significantly prolonged transgene expression. Although promising, they require a helper virus for production which has to be eliminated by purification.

The vast majority of pre-clinical and clinical trials using Ad vectors for somatic gene therapy and for cancer therapy or vaccination were based on Ad5 or Ad2. Both serotypes belong to subgenus C and are prevalent in the population. Therefore, a high degree of pre-existing immunity (in particular antibodies) against these serotypes is present within the population. This causes problems for gene therapy treatments and vaccination using subgenus C-based vectors as they may be neutralized by the antibodies and rapidly eliminated, and thus will not be therapeutically efficacious (Horwitz, 2001).

Different types of “therapeutic” adenoviruses and adenovirus vectors may be used. Ads may act as ‘oncolytic viruses’ (“onco” meaning cancer, “lytic” meaning “killing”), designed to infect and/or replicate in cancer cells, destroying these harmful cells and leaving normal cells largely unaffected (Dobbelstein, 2004). Oncolytic viruses utilize multiple mechanisms to kill cancer cells, e.g. apoptosis, cell necrosis or anti-angiogenesis. Once the virus infects the tumour cell, it compromises the cell's intrinsic defence mechanisms, giving the virus extra time to thrive. The virus then begins to replicate. Replication continues until the tumour cell can no longer contain the virus and eventually “lyses” (bursts). The tumour cell is destroyed and the newly created viruses are spread to neighbouring cancer cells to continue the cycle.

Rather than utilizing the intrinsic cytolytic effect of the mere Ad infection for direct killing of cancer cells, cytotoxic (proapoptotic or pronecrotic) genes can be incorporated into replication-deficient adenovirus vectors, which on expression in the cancer cells induce their death. Multiple systems have been explored. Apart from encoding directly cytotoxic gene products Ads may express enzymes that convert non-toxic substrates into toxic ones (Palmer et al., 2002).

A more indirect approach to eradicate cancer cells aims at stimulating the patient's own immune system to kill the tumour cells. A very promising approach relies on dendritic cells (DCs). DCs are professional antigen-presenting leukocytes which are present in almost all tissues of the body and are very effective in activating resting/naive T-cells (Banchereau et al., 2000). A large body of evidence shows that DCs loaded with tumour antigens can efficiently process and present tumour antigens to the immune system and thereby can induce effective immune responses against cancer. Various methods (peptide loading, RNA and DNA transfection, or infection) have been used to introduce antigens or their encoding genes into DCs (Schuler et al., 2003).

A prerequisite for successful therapy is high infection/transduction efficiency for the corresponding target tissue. In this regard, the limited efficiency of infection of certain target cells and tumours by the conventional subgenus C adenoviruses and vectors derived thereof can be a significant drawback (Kim et al., 2002). Such infection/transduction depends primarily upon the presence on the cell surface of the coxsackie adenovirus receptor (CAR), the primary receptor for subgenus C adenoviruses (Bergelson et al., 1997; Roelvink et al., 1998). This receptor is important for the initial attachment of these viruses to the target cell membrane. Subsequent interactions of the Ad penton base with certain cellular integrins and potential interactions with other Ad components contribute to the efficiency of Ad infections (Nemerow, 2000). Many tumour types but also DCs are reported to be relatively refractory to Ad5 and Ad2 infection and this phenotype often correlates with a low cell surface expression or a complete lack of CAR (Kim et al., 2002). For example, DCs generally lack CAR expression.

There is therefore a need for a more efficient transduction of target cells by adenoviral vectors.

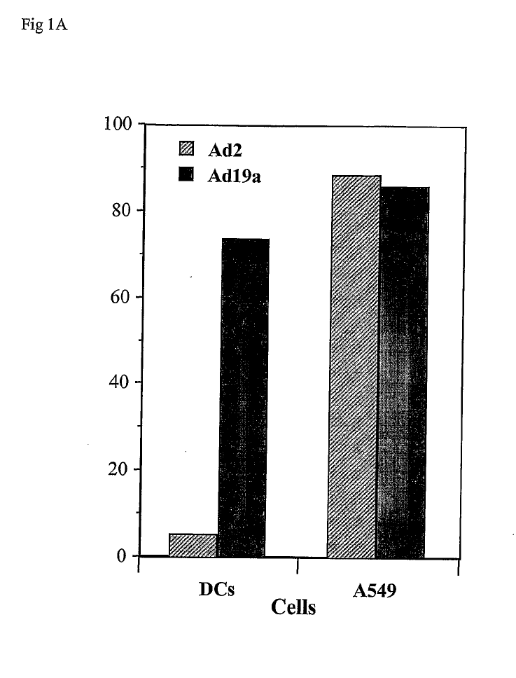

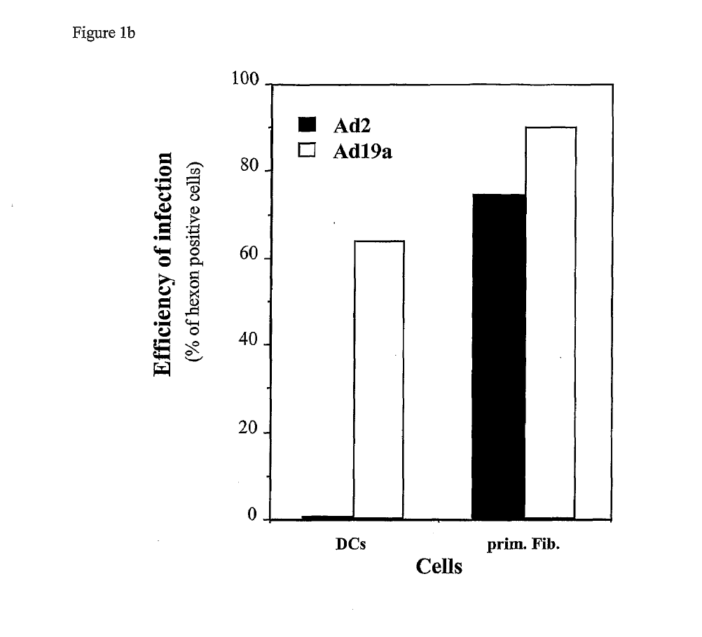

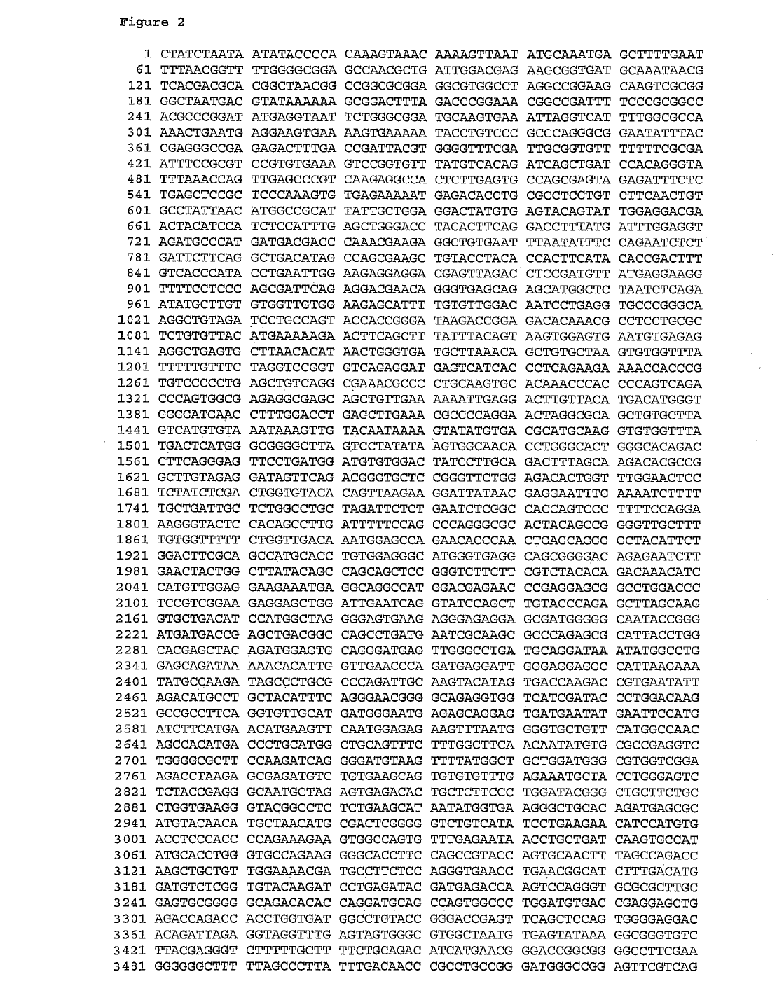

By screening various serotypes from different subgenera for their infection efficiency of DCs, we have identified adenovirus serotype Ad19a as being particularly efficient for DC infection (FIG. 1A). Quantitative FACS analysis monitoring adenovirus hexon expression indicated that more than 70% of DCs were successfully infected by Ad19a whereas less than 10% could be infected by Ad2. The infection efficiencies of the two viruses was not significantly different when the lung epithelial cell line A549 was examined. A similar picture was seen when infection efficiency in primary human DCs was compared with that in primary foreskin fibroblasts SeBu (FIG. 1B). Again a drastic difference between the two viruses was noted in infecting DCs while infection efficiency was similar for the fibroblasts. This shows that the two viruses are similarly effective for infection of fibroblasts and lung epithelial cells whereas they differ dramatically in their efficiency to infect DCs. As the DCs used did not express CAR (data not shown), Ad19a does not appear to require CAR for infection as opposed to subgenus C Ads. Thus, Ad19a, and possibly other Ads of subgenus D, target different surface structures and are therefore likely to exhibit a very different target specificity for cells (Arnberg et al., 2000; Wu et al., 2001). This feature may be extremely useful for efficient targeting/transduction of Ads and Ad-derived vectors of DCs, leukocytes in general, and various other tissues (e.g. eye tissues) and tumour cells. Even for cells that can be transduced by conventional vectors, Ad19a-derived vectors may be beneficial, in that lower amounts of viruses or vectors may be needed for efficient infection and efficient expression of the transgene, thus reducing potential toxicity and immunogenicity.

We also describe a new method to construct and manipulate recombinant DNA containing genomic adenoviral nucleic acid in bacteria and in vitro, which is generally applicable to all adenovirus genomes. A reason for the relative small number of cloned Ad genomes is the rather laborious procedure for cloning of new serotypes. Furthermore, new serotypes are difficult or impossible to clone with the standard methods and vectors. We have developed new methods for convenient cloning and genetic manipulation of adenoviruses which are herein disclosed as well as the adenoviruses manipulated by said method.

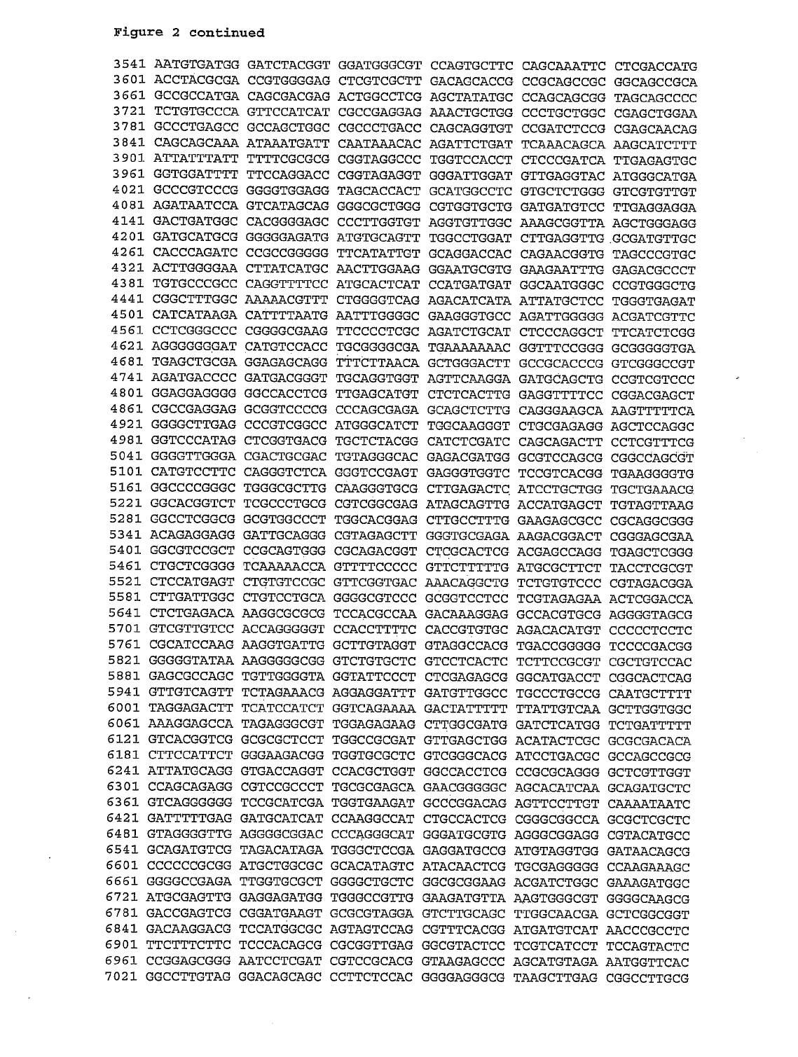

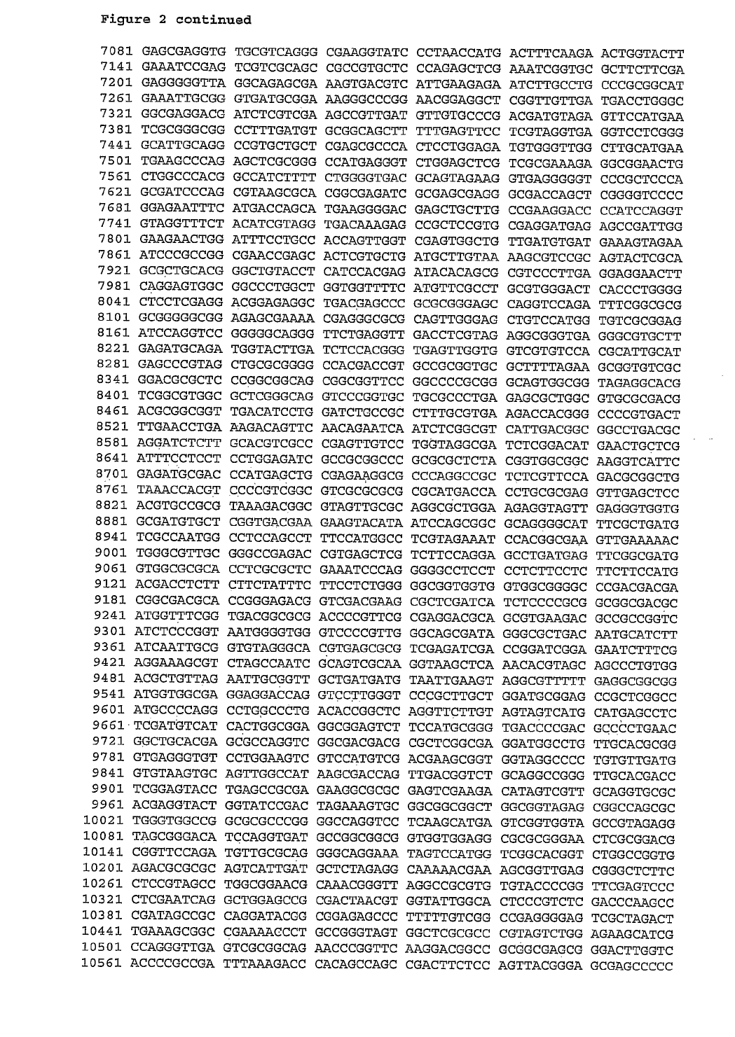

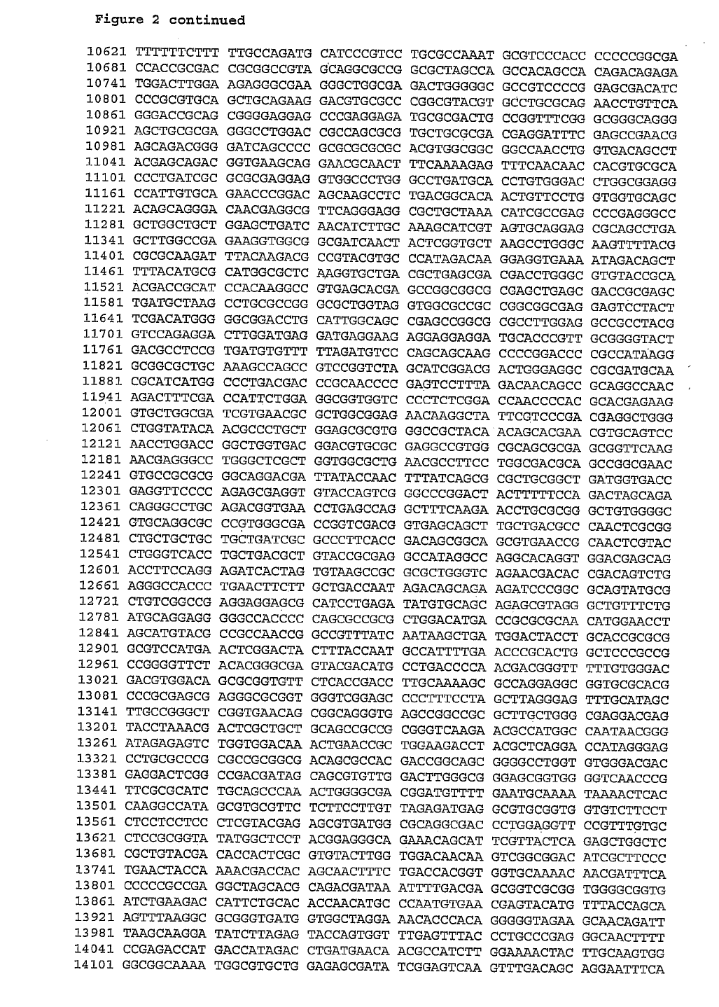

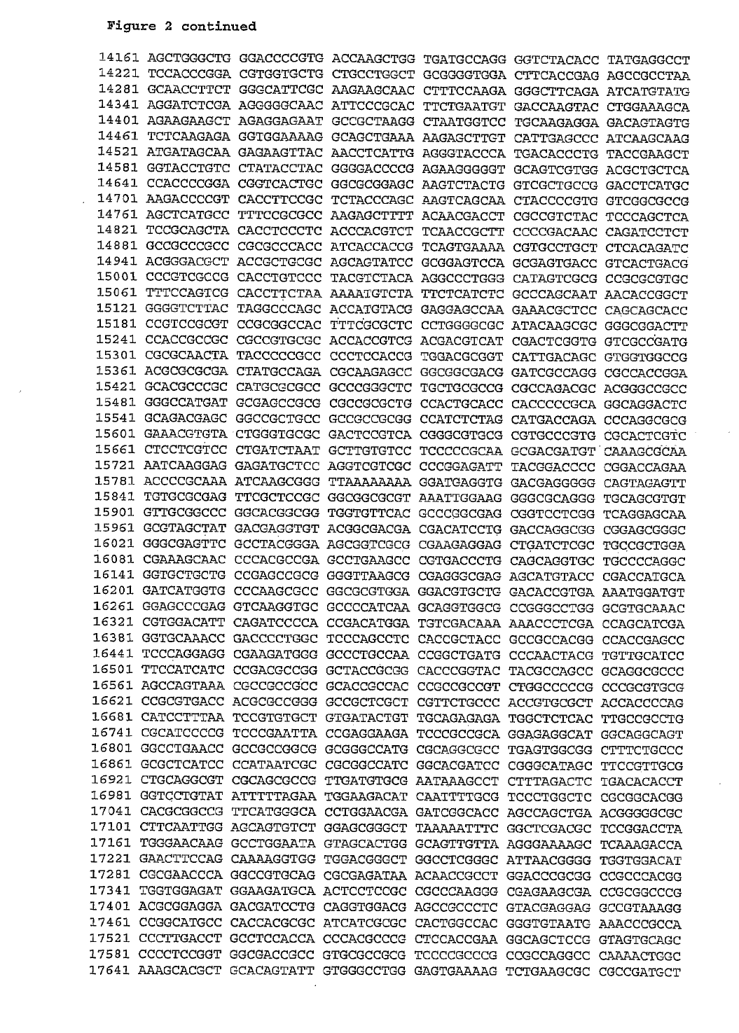

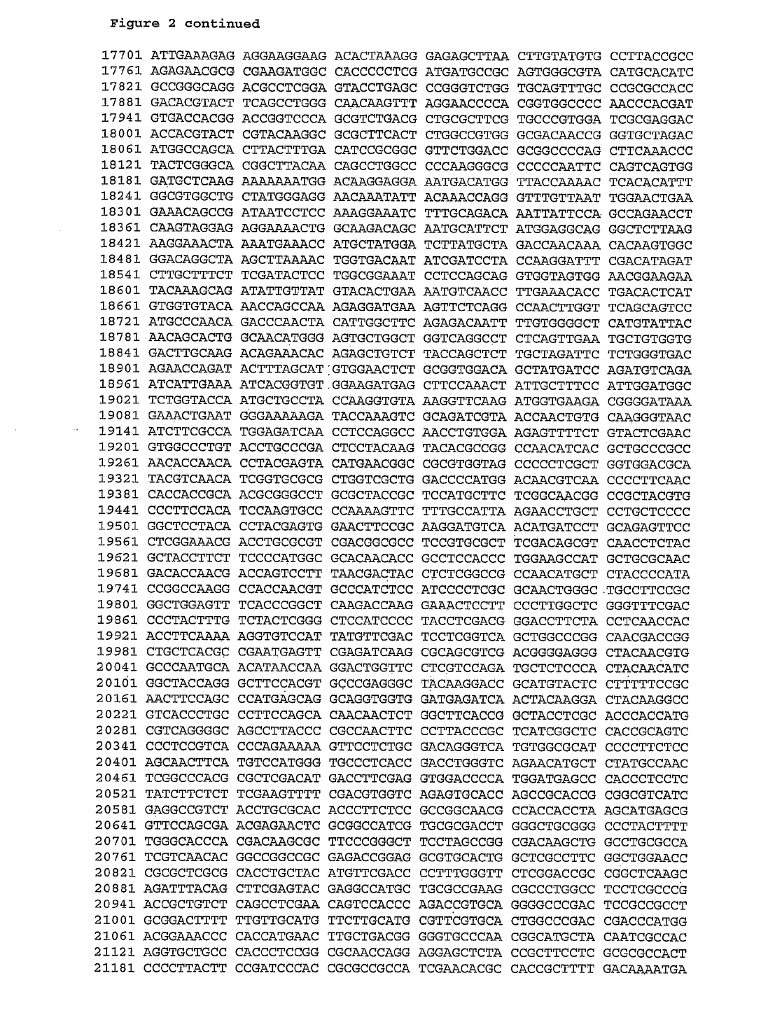

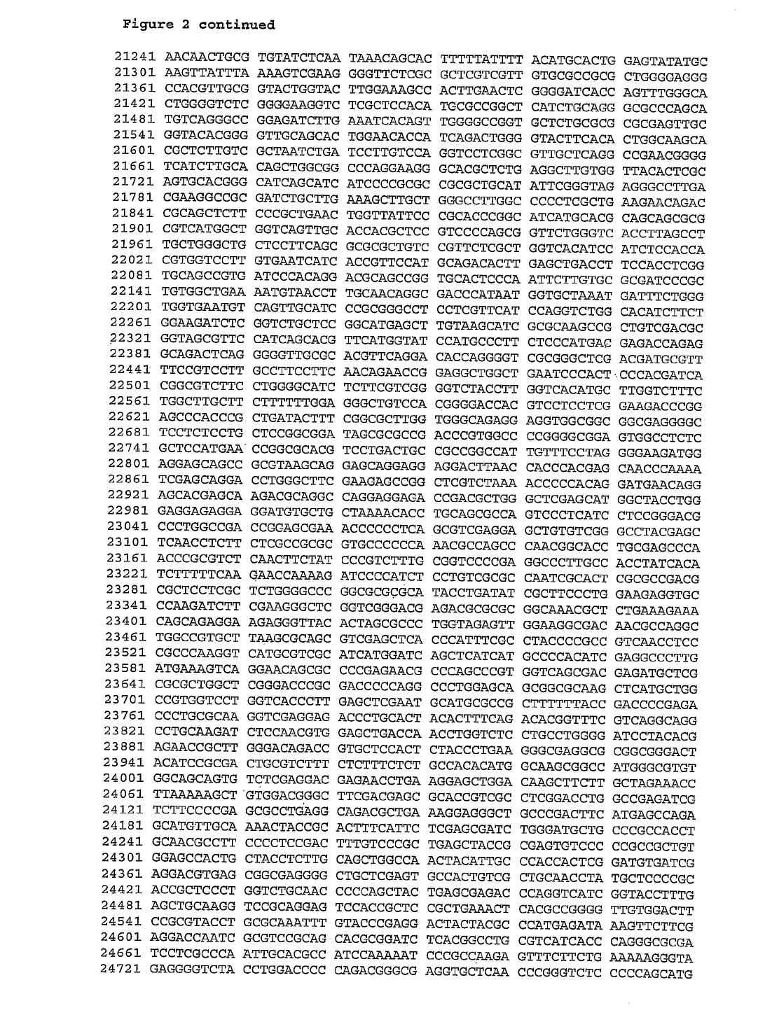

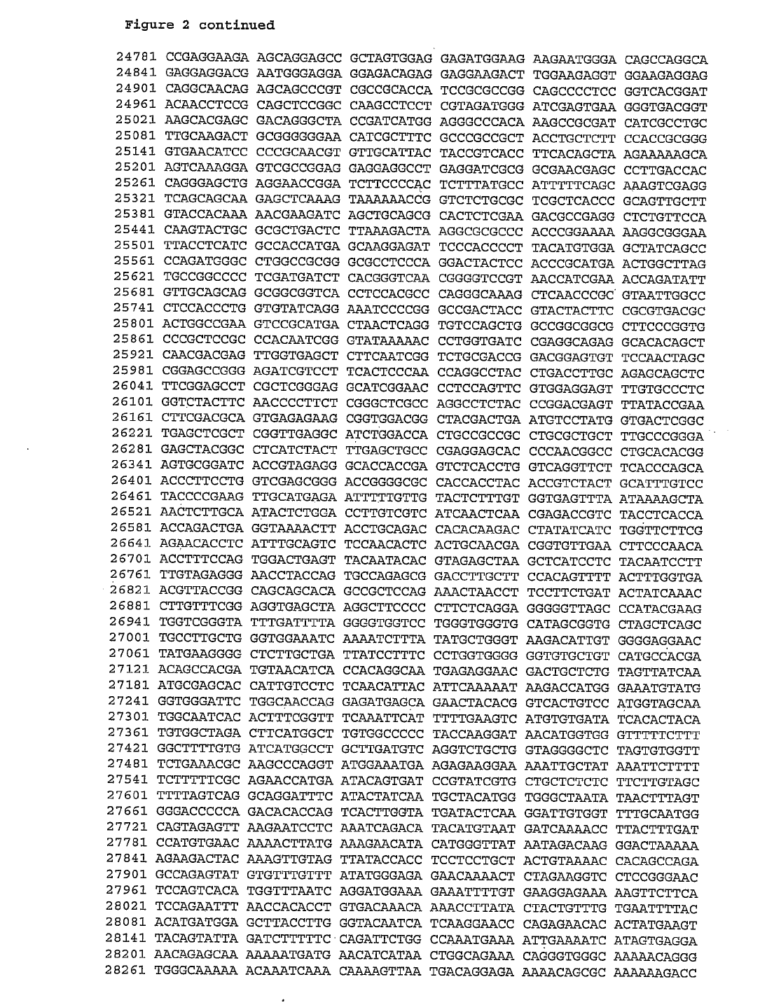

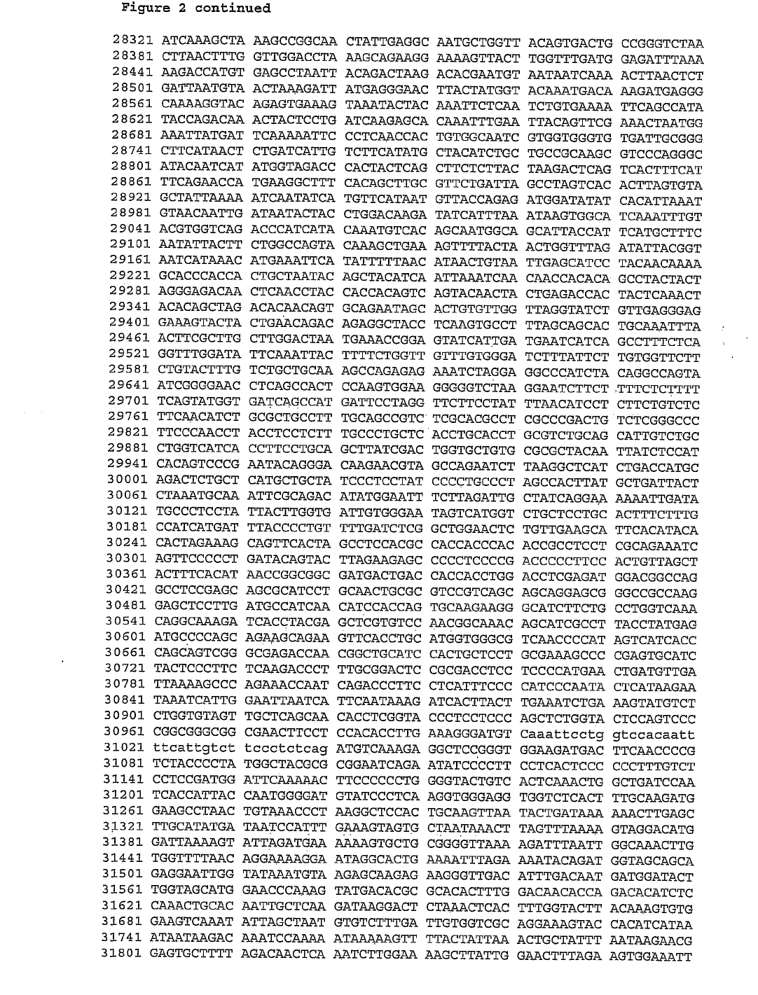

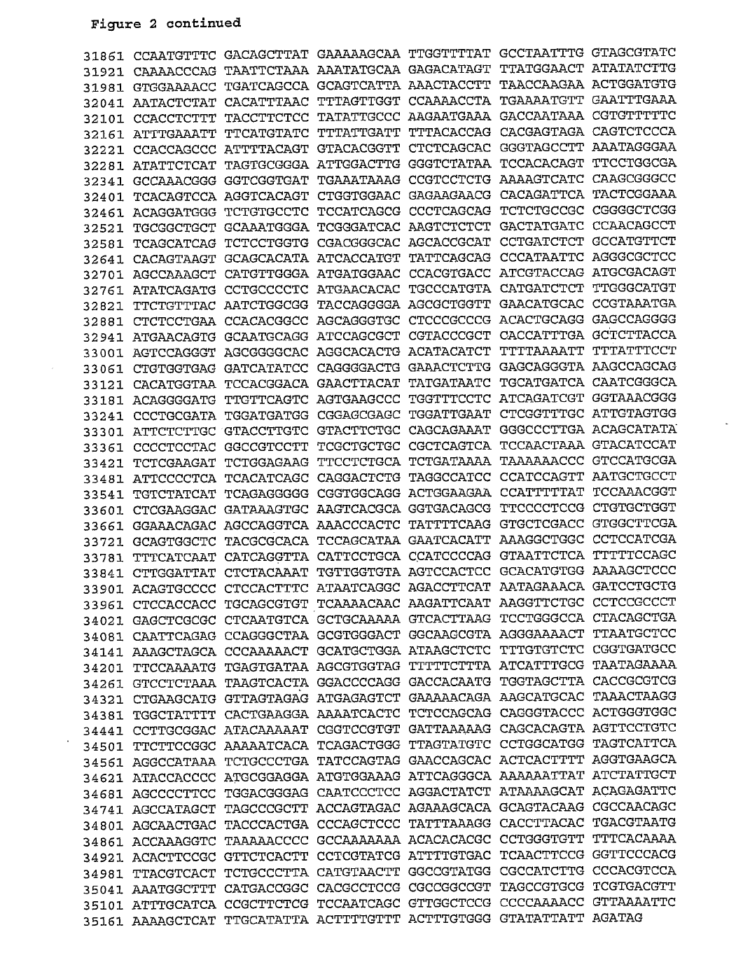

According to a first aspect of the invention there is provided an isolated adenovirus wherein the genome of said adenovirus comprises the nucleic acid sequence as shown in FIG. 2, or a variant adenovirus wherein said adenovirus genome is modified by the addition, deletion or substitution of at least one nucleotide base and hybridises to the sequence shown in FIG. 2. Preferably said hybridisation conditions are stringent.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology-Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (allows sequences that share at least 90% identity to hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (allows sequences that share at least 80% identity to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (allows sequences that share at least 50% identity to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said adenovirus is of subgenus D. Preferably said adenovirus is Ad19a.

In an alternative preferred embodiment of the invention said adenovirus is Ad19p.

In a further alternative preferred embodiment of the invention said adenovirus belongs to the adenovirus group that causes epidemic keratoconjunctivitis, and thus may be Ad8, Ad19a or Ad37.

In a yet further alternative preferred embodiment of the invention said adenovirus is selected from the group consisting of: Ad9, 10, 13, 15, 17, 20, 22-30, 32, 33, 36-39, 42-47, 51.

(Please see De Jong et al., 1999; or Shenk, 2001 for a description of adenovirus types)

In a preferred embodiment of the invention the adenovirus genome is modified within the E1A and/or E1B genes to generate an Ad or an Ad vector.

In a preferred embodiment of the invention the adenovirus genome is modified by the inclusion of at least one heterologous nucleic acid molecule.

In a further preferred embodiment the genome of the adenovirus is adapted for eukaryotic expression of said heterologous nucleic acid molecule.

Typically said adaptation includes, by example and not by way of limitation, the provision of transcription control sequences (promoter sequences) that mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis-acting nucleic acid sequences often found 5′ to the transcription initiation site of a gene (enhancers can also be found 3′ to a gene sequence or even located in intronic sequences and are therefore position-independent). Enhancers function to increase the rate of transcription of the gene to which the enhancer is linked. Enhancer activity is responsive to trans-acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues.

Promoter elements also include the so-called TATA box and RNA polymerase initiation selection sequences which function to select a site of transcription initiation. These sequences also bind polypeptides which function, inter alia, to facilitate transcription initiation selection by RNA polymerase.

Adaptations which facilitate the expression of Adenovirus-encoded genes include the provision of transcription termination/polyadenylation sequences. This also includes the provision of internal ribosome entry sites (IRES) that function to maximise expression of Adenovirus-encoded genes arranged in bicistronic or multi-cistronic expression cassettes.

These adaptations are well known in the art. There is a significant amount of published literature with respect to expression vector construction and recombinant DNA techniques in general. Please see, (Sambrook et al., 1989) and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc.(1994).

The specificity and safety of gene therapy is enhanced by limiting the expression of the gene in specific tissues and/or cells. Preferably, therefore the expression of the heterologous nucleic acid is controlled by a tissue and/or cell specific and/or cancer specific promoter. Cancer-specific promoters include for example breast, prostate, and melanoma-specific promoters. Also, DC-specific promoters have been identified (Ross R, 2003).

In a further preferred embodiment of the invention the heterologous nucleic acid molecule encodes a therapeutic agent, which when expressed in a target cell produces a therapeutic effect.

The heterologous nucleic acid molecule may encode tumour suppressor genes, antigenic genes, cytotoxic genes, cytostatic genes, pro-drug activating genes, apoptotic genes, pharmaceutical genes or anti-angiogenic genes (Kanerva and Hemminki, 2004; St George, 2003). The adenovirus of the present invention may be used to produce one or more therapeutic transgenes, either in tandem through the use of IRES elements or through independently regulated promoters.

Preferably the therapeutic agent is a polypeptide.

Preferably the heterologous nucleic acid encodes an antigenic polypeptide. Examples of antigenic polypeptides include carcino-embryonic antigen (CEA), p53 (as described in Levine, A. PCT International Publication No. WO94/02167 published Feb. 3, 1994) or HIV antigens, env, gag, pol or Tat. In order to facilitate immune recognition, parts of the antigenic polypeptide or sequences representing antigenic epitopes may be expressed either alone or fused to those of other antigens. Selected antigens may be presented by MHC class I and MHC class II molecules, as well as by non-classical MHC molecules.

Preferably, the antigenic polypeptide is derived from a tumour cell-specific antigen, ideally a tumour rejection or a tumour associated antigen (TAA). Tumour rejection antigens, also called tumour specific transplantation antigens (TSTA), are well known in the art and include, by example and not by way of limitation, the MAGE, BAGE, GAGE and DAGE families of TAAs (van der Bruggen et al., 2002).

It has been known for many years that tumour cells produce a number of tumour cell-specific antigens, some of which are presented at the tumour cell surface. These are generally referred to as tumour rejection antigens and are derived from larger polypeptides referred to as tumour rejection antigen precursors. Generally, tumour rejection antigens are presented via HLA class I or class II molecules to the host's T cells. Other tumour-specific antigens may be presented by CD1 molecules or may directly activate certain cells of the immune system, e.g natural killer (NK) cells or NKT cells. Examples for the latter are MHC-like tumour-specific stress molecules, such as MICA-MICE. In general, the immune system recognises these abnormally expressed molecules as foreign or abnormal and destroys cells expressing these antigens. If a transformed cell escapes detection and becomes established, a tumour develops. Various vaccines have been developed based on dominant tumour rejection antigens to provide individuals with a preformed defence to the establishment of a tumour.

In a preferred embodiment of the invention the therapeutic agent is a tumour rejection antigen or a TAA.

In a still further preferred embodiment of the invention said heterologous nucleic acid encodes a cytotoxic agent. Said cytotoxic agent may be selected from the group consisting of; pseudomonas exotoxin; ricin toxin; diptheria toxin and the like.

In a further preferred embodiment of the invention said heterologous nucleic acid encodes a polypeptide with cytostatic activity thereby inducing cell-cycle arrest. Examples of such cytostatic genes include p21, the retinoblastoma (Rb) gene, the E2F-Rb gene, genes encoding cyclin dependent kinase inhibitors such as P16, p15, p18 and p19, the growth arrest specific homeobox (GAX) gene as described in Branellec, et al. (PCT Publication WO97/16459 published May 9, 1997 and PCT Publication WO96/30385 published Oct. 3, 1996).

In a still further preferred embodiment of the invention said heterologous nucleic acid encodes a pharmaceutically active polypeptide. Preferably said pharmaceutically active polypeptide is a cytokine. The term “cytokine gene” refers to a nucleotide sequence, the expression of which in a cell produces a cytokine. Examples of such cytokines include GM-CSF, the interleukins, especially IL-1, IL-2, IL-4, IL-5, IL-12, IL-10, IL-15, IL-19, IL-20, interferons of the α, β, and γ subtypes, and members of the tumour necrosis factor family.

In a further preferred embodiment of the invention said pharmaceutically active polypeptide is a chemokine.

The term “chemokine gene” refers to a nucleotide sequence, the expression of which in a cell produces a chemokine. The term chemokine refers to a group of structurally related low-molecular weight cytokines secreted by cells having mitogenic, chemotactic or inflammatory activities. They are primarily cationic proteins of 70 to 100 amino acid residues that share four conserved cysteines. These proteins can be sorted into two groups based on the spacing of the two amino-terminal cysteines (Mantovani et al., 2004). In the first group, the two cysteines are separated by a single residue (C-x-C), while in the second group, they are adjacent (C-C). Examples of member of the ‘C-x-C’ chemokines include but are not limited to platelet factor 4 (PF4), platelet basic protein (PBP), interleukin-8 (IL-8), IP-10, melanoma growth stimulatory activity protein (MGSA), BCA-1, I-TAC, SDF-1 etc. and pre-B cell growth stimulating factor (PBSF). Examples of members of the ‘C-C’ group include but are not limited to monocyte chemotactic protein 1 (MCP-1), MCP-2, MCP-3, MCP-4, macrophage inflammatory protein 1 α (MIP-1-α), MIP-1-β, MIP3α, MIP3β, MIP-5/HCC-2, RANTES, thymus and activation-regulated chemokine (TARC), eotaxin, I-309, human protein HCC-1 and HCC-3 (Balkwill, 2004).

In a still further preferred embodiment of the invention said polypeptide is an antibody or active binding fragment thereof. Preferably said antibody or binding fragment is a monoclonal antibody. Preferably said fragment is a Fab fragment or a single chain antibody variable fragment.

In a further preferred embodiment of the invention said heterologous nucleic acid encodes a tumour suppressor polypeptide. Preferably said tumour suppressor polypeptide is p53.

A tumour suppressor gene is a gene encoding a protein that suppresses tumour formation, thus it is a gene that normally prevents unlimited cell division. When both copies of the gene are lost or mutated the cell is transformed to a cancerous phenotype. Examples are the p53, retinoblastoma and Wilm's tumour genes.

In a further preferred embodiment of the invention said heterologous nucleic acid encodes a polypeptide which induces apoptosis or other forms of cell death. Examples of pro-apoptotic genes include p53, the adenovirus E4or f4 gene, p53 pathway genes, genes encoding caspases or proapoptotic Bc1-2 family members, proapoptotic ligands (TNF, FasL, TRAIL) and/or their receptors (TNFR, Fas, TRAIL-R1, TRAIL-R2). A cytolytic function has also been ascribed to the E3/11.6 K protein of subgenus C adenoviruses that may therefore be incorporated as a therapeutic gene (Doronin et al., 2000).

In a further preferred embodiment of the invention the polypeptide is a pro-drug activating polypeptide.

The term “pro-drug activating genes” refers to nucleotide sequences, the expression of which, results in the production of proteins capable of converting a non-therapeutic compound into a therapeutic compound, which renders the cell susceptible to killing by external factors or causes a toxic condition in the cell. An example of a prodrug activating gene is the cytosine deaminase gene. Cytosine deaminase converts 5-fluorocytosine to 5 fluorouracil, a potent antitumour agent. The lysis of the tumour cell provides a localized burst of cytosine deaminase capable of converting 5 FC to 5 FU at the localized point of the tumour resulting in the killing of many surrounding tumour cells. This results in the killing of a large number of tumour cells without the necessity of infecting these cells with an adenovirus (the so-called bystander effect). Additionally, the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product become susceptible to selective killing by the administration of ganciclovir may be employed. (Please see Palmer et al., 2002 for a description of various prodrug activating enzymes).

In a further preferred embodiment of the invention the polypeptide has anti-angiogenic activity

The term “anti-angiogenic” genes refers to a nucleotide sequence, the expression of which results in the extracellular secretion of anti-angiogenic factors. Anti-angiogenesis factors include angiostatin, inhibitors of vascular endothelial growth factor (VEGF) such as Tie 2 (as described in PNAS (USA) (1998) 95:8795-8800), endostatin. Also see, Kerbel and Folkman, 2002)

In a further preferred embodiment of the invention the therapeutic molecule is an antisense nucleic acid molecule.

Antisense technology emerged in the 1980s as a way to target the RNA molecules rather than the proteins that they encode. Antisense technology does not rely on small molecule therapeutics to target RNA targets, but instead employs modified strands of DNA that can bind to specific RNA sequences. When the modified DNA strands bind to the targeted RNA, the RNA can no longer be translated into protein. As a result, if a disease is characterized by the excessive production of a particular protein product, targeting the RNA which encodes the protein and preventing their translation may be a safer, more viable, and more effective form of treatment.

In a further preferred embodiment of the invention the therapeutic molecule is an inhibitory RNA (RNAi) or a small inhibitory RNA (siRNA). SiRNA molecules are RNA molecules that function to bind to specific cellular target molecules, thereby inducing the specific degradation of the targeted mRNA. As a consequence, synthesis of specific proteins can be greatly diminished. This therefore allows the specific elimination of expression of certain genes (Dykxhoorn DM, 2003). Systems for both transient and permanent expression of siRNA have been developed which may be incorporated into the said Ad or Ad vector (Brummelkamp et al., 2002). Typically siRNAs are small double stranded RNA molecules that vary in length from between 10-100 base pairs in length although large siRNA's e.g. 100-1000 bp can be utilised. Preferably the siRNAs are about 21 to 23 base pairs in length. Alternatively, short hairpin RNAs (shRNAs) may be designed based on small, non-coding microRNA molecules with a ‘hairpin’ secondary structure. Incorporation of such synthetic elements in Ads can be used to selectively silence gene expression by RNA interference (RNAi), similar to siRNAs.

In a further preferred embodiment of the invention the therapeutic molecule is a ribozyme. In this case, the expressed RNA has enzymatic activity, destroying by way of their design selected cellular mRNAs.

Typically, when using adenovirus-based vectors for gene therapy, the virus has to be modified to eliminate or minimise the disease-causing potential by rendering the virus replication-deficient. Typically, such a modification involves the deletion of the E1 region genes. Thus, in a further preferred embodiment of the invention the said adenovirus is made replication-deficient, preferably the adenovirus is E1 negative.

In addition, the adenovirus virus vector may harbour deletions within the E3 region or may be deficient in one or more E3 functions. Moreover, certain E3 genes, individual or as a whole, may be replaced by other “therapeutic” genes, including genes encoding antigenic proteins for vaccination, or may be selectively overexpressed, e.g. to interfere with particular immune finctions or increase lysis.

If a protein is being utilised for therapeutic purposes it is often desirable to be able to confirm and visualise its expression. This is typically achieved by the use of protein tags. The DNA sequence that codes for the therapeutic protein is tagged by fusing it to the sequence of another protein that can be easily detected. When the organism expresses the therapeutic protein, the protein “tags” are also produced.

Proteinaceous fluorophores are known in the art. Green fluorescent protein, GFP, is a spontaneously fluorescent protein isolated from coelenterates, such as the Pacific jellyfish, Aequoria victoria. Its role is to transduce, by energy transfer, the blue chemiluminescence of another protein, aequorin, into green fluorescent light. GFP can function as a protein tag, as it tolerates N- and C-terminal fusions to a broad variety of proteins many of which have been shown to retain native function. Most often it is used in the form of enhanced GFP in which codon usage is adapted to the human code. Other proteinaceous fluorophores include yellow, red and blue fluorescent proteins.

In a further preferred embodiment of the invention the adenovirus further comprises a protein tag. Preferably the protein tag is a fluorescent protein. Even more preferably the fluorescent protein is green fluorescent protein.

In an even further preferred embodiment of the invention the adenovirus genome sequence is modified to encode green fluorescent protein, a derivative thereof or another fluorescent protein.

The fluorescent proteins may be expressed independently from other Ad proteins or heterologous sequences using specific promoters, enhancers and polyadenylation signals, as discussed above. It can be used to conveniently monitor transduction efficiency of vectors. Other marker proteins, such as β-galactosidase, may be expressed in the viral genome to quantitate the efficiency of transduction/infection.

It will be readily apparent to those of skill in the art that there may be modifications and/or deletions to the above referenced heterologous nucleic acid molecules so as to encode functional sub-fragments of the wild type protein which may be readily adapted for use in the practice of the present invention. For example, the reference to the p53 gene includes not only the wild type protein but also modified p53 proteins. Examples of such modified p53 proteins include modifications to p53 to increase nuclear retention, such as the deletion of the calpain consensus cleavage site (Kubbutat and Vousden (1997) Mol. Cell. Biol. 17:460-468, modifications to the oligomerization domains (as described in Bracco, et al. PCT published application WO97/0492 or U.S. Pat. No. 5,573,925, etc.).

It will be readily apparent to those of skill in the art that the above therapeutic genes may be localized to particular intracellular locations by inclusion of a targeting moiety, such as a signal peptide, an endoplasmic reticulum retention signal, other transport motifs or a nuclear localization signal (NLS). In other instances, targeting signals may be included that allow efficient secretion of the therapeutic gene.

In a further preferred embodiment of the invention the adenovirus is further modified to generate a high capacity adenovirus vector (HCAdV).

These viruses are devoid of any adenovirus genes and essentially contain only the inverted terminal repeats and the DNA packaging signals. Typically they are also referred to as “gutted” or “gutless” adenoviruses (Volpers and Kochanek, 2004). In animal models these types of viruses show profoundly improved persistence of transgene expression. However, production of HCAdV requires the co-infection with a modified helper adenovirus, in this case a modified helper Ad19a.

According to a further aspect of the invention there is provided a chimeric adenovirus comprising a first nucleic acid comprising an adenovirus nucleic acid, or part thereof, and at least one second nucleic acid comprising an adenovirus nucleic acid, according to the invention, or part thereof that is different from said first adenoviral nucleic acid.

The word “chimeric” denotes an adenonvirus genome that combines advantageous properties of one adenonvirus with that of another, different adenovirus. For example, and not by way of limitation, the targeting specificity of Ad19a or other members of subgenus D may be transferred to, the Ad5 or Ad2 genomes or the relevant Ad2 and Ad5 vectors whereby the Ad5 fiber or parts thereof (e.g. the fiber knob, the shaft or penton interacting sequences) are replaced by the fiber, or parts thereof of Ad19a or other members of subgenus D. These Ad5-Ad19a chimeric viruses or vectors may at least in part transfer the Ad19a targeting specificity on to a known vector.

According to a further aspect of the invention there is provided a cell comprising an adenovirus according to the invention.

Preferably the cell is a prokaryotic cell. Alternatively, the cell is a eukaryotic cell.

In a preferred embodiment the cell is a mammalian cell, preferably a human cell.

In a preferred embodiment of the invention said cell expresses low levels of coxsackie adenovirus receptor (CAR). Preferably said cell does not express detectable levels of CAR.

In a preferred embodiment of the invention said cell is a cell derived from ocular tissue. Preferably said cell is derived from corneal tissue; conjunctiva tissue; retinal tissue, for example retinal pigment epithelial cells.

In a further preferred embodiment of the invention said cell is derived from lung tissue. Preferably said cell is derived from differentiated lung epithelial tissue or bronchial epithelial tissue.

In a still further preferred embodiment of the invention said cell is a haematopoietic cell.

Preferably, said cell is a haematopoietic stem cell, for example a CD34 expressing cell.

Preferably said haematopoietic cell is a leukocyte, for example a lymphocyte. Even more preferably the cell is an antigen presenting cell, preferably a dendritic cell.

In a yet further preferred embodiment of the invention said cell is an endothelial cell.

In a preferred embodiment of the invention said cell is a muscle cell. Preferably said muscle cell is selected from the group consisting of: cardiac muscle, striated muscle or smooth muscle.

In a further preferred embodiment of the invention said cell is a neuron, for example a brain neuron.

In another preferred embodiment the cell is a cancer cell. Preferably said cancer cell is a cell that expresses low levels of coxsackie adenovirus receptor. Preferably said cancer cell does not express detectable levels of coxsackie adenovirus receptor.

In a preferred embodiment of the invention said cancer cell is a cancer cell of lymphoid origin, for example a chronic lymphocytic leukaemic cell.

In a further preferred embodiment of the invention said cancer cell is a glioma cell, for example a brain glioma cell. Glioma cells can be primary or secondary glioma cells.

In a further preferred embodiment of the invention said cancer cell is an androgen resistant prostate cancer cell.

In a yet further preferred embodiment of the invention said cancer cell is a melanoma cell.

In a preferred embodiment of the invention said cancer cell is bladder cancer cell.

In a preferred embodiment of the invention said cancer cell is an ovarian cancer cell.

In a further preferred embodiment of the invention said cancer cell is a colorectal cancer cell.

In a further preferred embodiment of the invention said cancer cell is a cervical cancer cell.

According to a further aspect of the invention there is provided a pharmaceutical composition comprising the adenovirus or cell according to the invention.

In a preferred embodiment of the invention said composition further comprises a second therapeutic agent.

Preferably said second therapeutic agent is a chemotherapeutic agent.

According to a further aspect of the invention there is provided the use of an adenovirus according to the invention for the manufacture of a medicament for use in the treatment of cancer.

According to a yet further aspect of the invention there is provided a method of treatment of an animal, preferably a human, comprising the administration of a therapeutically effective amount of the adenovirus according to the invention. Preferably the method of treatment is for cancer.

It will be apparent that the said adenovirus vector(s) may equally be useful for other treatments, for example, for vaccinations (e.g. against infectious diseases), conventional gene therapy, for highly efficient protein expression or in the context of iRNA for depletion of protein expression (see above siRNA etc.) as the adenoviral vector according to the invention expresses protein approximately 10 fold higher than for example, Ad5 based vectors.

In a preferred embodiment of the invention the adenovirus-mediated gene therapy is combined with conventional treatment of cancer using, for example cytostatic drugs. In many cases, the combined treatment improved the success and allowed to reduce the concentration of the drug and/or the amount of virus vector.

According to a second aspect of the invention there is provided a method to construct recombinant adenoviral genomic nucleic acid comprising the steps of:
i) providing a preparation comprising a vector and an adenoviral genome, or part thereof, wherein the vector, or adenoviral genome, is adapted by the provision of nucleic acid sequence motifs which allow the recombination of the genome with the vector;
ii) transforming the vector and said adapted adenoviral genome into a bacterial cell wherein the bacterial cell is adapted to induce the recombination of said vector with said genome; and optionally
iii) purifying the recombinant vector containing the adenoviral genome and excising the adenoviral genome from the vector.

In a preferred method of the invention the restriction enzyme digested recombinant vector is transfected into a permissive cell.

According to a further aspect of the invention there is provided a method to construct recombinant adenoviral genomic nucleic acid comprising the following steps of:
i) providing a preparation comprising a bacterial vector comprising the left and the right termini of an adenovirus genome joined to the vector sequence by nucleic acid sequence motifs which allow in vitro excision of said genome;
ii) providing a preparation comprising a transposon-labelled adenovirus genomic nucleic acid;
iii) providing a preparation comprising bacterial cells carrying said vector and adapted to induce recombination between said vector and said transposon-labelled adenovirus genomic nucleic acid;
iv) transforming said bacterial cells with said transposon-labelled adenoviral genomic nucleic acid;
v) isolating bacteria carrying the recombinant comprising the vector and the said transposon-labelled adenoviral nucleic acid;
vi) excision of the said transposon from the said recombinants; and optionally
vii) purifying said recombinants and excise the adenovirus genome allowing reconstitution of said adenovirus by transfecting permissive cells, e.g. 293 cells.

In a preferred method of the invention said adenovirus is adenovirus 19a.

In a further method of the invention said nucleic acid sequence motifs for excision are recognition sequences of restriction endonucleases which do not cut the said adenovirus genome. Preferably said motifs are located adjacent to the inverted terminal repeats (ITRs) of said adenoviral genome that are used for recombination.

In a further method of the invention said nucleic acid sequence motifs for excision are PacI sequence motifs. In a further method of the invention said vector is a bacterial artificial chromosome (BAC).

In a yet further preferred method of the invention said bacterial adaptation is the provision of a cell that expresses phage recombination polypeptides, preferably the λ phage recombination polypeptides αβγ.

In a further preferred method of the invention for recombination and the generation of the adenoviral genomic nucleic acid said Ad genome is contacted with a nucleic acid molecule comprising a transposon (Tn) to form a transposon-containing adenoviral genomic nucleic acid. Preferably said transposon includes a nucleic acid molecule comprising a nucleic acid sequence which encodes a selectable marker in bacteria.

In a yet further preferred method of the invention said transposon-containing adenoviral genomic nucleic acid is transformed into a bacterial cell adapted to allow the recombination of said transposon-containing adenoviral genomic nucleic acid into said vector.

In a preferred method of the invention said transformed bacterial cell is cultured in medium which includes an agent which selects for said transformed bacterial cell. Preferably said agent is an antibiotic.

In a still further preferred method of the invention said transposon is subsequently excised from said recombinant transposon-containing adenoviral genomic nucleic acid.

In a preferred method of the invention said Tn is excised by contacting said Tn with a transposase. Preferably said transposase is derived from the TnsABC complex. This will generate an adenoviral genomic sequence lacking any remaining operational sequences.

According to a further aspect of the invention there is provided a method for the production of mutated adenoviral genomic nucleic acid comprising the steps of:
i) providing a preparation comprising a bacterial cell transformed with; a) a vector comprising adenoviral genomic nucleic acid and b) a nucleic acid molecule comprising a transposon and adenoviral nucleic acid wherein the adenoviral nucleic acid associated with said transposon is modified by addition, deletion or substitution of at least one nucleotide base and further wherein said transposon includes a nucleic acid molecule which encodes a selectable marker;
ii) growing said bacterial cells in conditions which allow for the selection of transformants which include vector/transposon recombinant nucleic acid molecules;
iii) isolating said recombinant nucleic acid molecule from said bacterial cell.

In a preferred method of the invention the isolated nucleic acid molecule is transfected into a permissive cell.

In a preferred method of the invention said transposon-containing nucleic acid is provided with at least 36 base pairs that are homologous to said adenovirus nucleic acid

In a preferred method of the invention the homologous nucleic acid sequences are organized to create 3 base pair direct repeats at the ends joined to the transposon.

In a preferred method of the invention said recombinant nucleic acid molecule is contacted with a transposase which excises said transposon from said adenoviral nucleic acid to introduce into said adenoviral genomic nucleic acid at least one mutation.

According to a further aspect of the invention there is provided a method for the production of mutated adenoviral genomic nucleic acid comprising the steps of:
i) providing a preparation comprising a bacterial cell transformed with a vector comprising adenoviral genomic nucleic acid and adapted to induce ET recombination;
ii) providing a preparation comprising a bacterial cell transformed with a vector comprising adenoviral genomic nucleic acid and a nucleic acid molecule comprising a transposon wherein said transposon nucleic acid sequence has been modified by addition at both ends of the transposon sequences of at least 36 base pairs homologies to the targeted adenovirus nucleic acid wherein the added nucleic acid sequences are organized such as to create 3 base pair direct repeats at the ends joined to the transposon and further wherein the modified transposon includes a nucleic acid molecule which encodes a selectable marker;
iii) transforming said modified transposon nucleic acid into said bacterial cell(s); and
iv) growing said bacterial cells in conditions which allow for the selection of transformants which include vector/transposon recombinant nucleic acid molecules;
v) isolating said recombinant nucleic acid molecule from said bacterial cell(s)
vi) excision of the said transposon from the said recombinants resulting in deletion, insertion or replacement of at least one base pair of said adenovirus genome; and optionally
vii) purifying said recombinants and excise the adenovirus genome allowing reconstitution of said adenovirus by transfecting permissive cells.

In a preferred method of the invention said recombinant nucleic acid molecule is contacted with a transposase which excises said transposon from said adenoviral nucleic acid to introduce into said adenoviral genomic nucleic acid at least one mutation.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following Figures:

FIG. 1: Highly efficient infection of Dendritic cells with Ad19a as compared to Ad2.
(A) Immature DCs were generated from peripheral blood monocytes by incubation with GM-CSF and IL-4 for 7 days. DCs were harvested, washed and infected with Ad2 (200 PFU/cell) and Ad19a (50 PFU/cell). 48 h later cells were processed for flow cytometry (FACS) by intracellular staining for the Ad hexon protein using mAb 2Hx-2. In parallel, the lung epitheloid cell line A549 was infected for 24 h and subsequently stained for FACS analysis. (B) In a similar set of experiments, the infection efficiency of the two viruses in DCs was compared with that in primary untransformed fibroblasts (SeBu). FACS analysis was done 44 h post infection. Again, while the infection efficiency in fibroblasts was highly efficient for both viruses (Ad2: 75%; Ad19a: 90%), only Ad19a was able to infect DCs efficiently.

FIG. 2 shows the sequence of the Ad19a genome from the left to the right inverted terminal repeat (ITR).

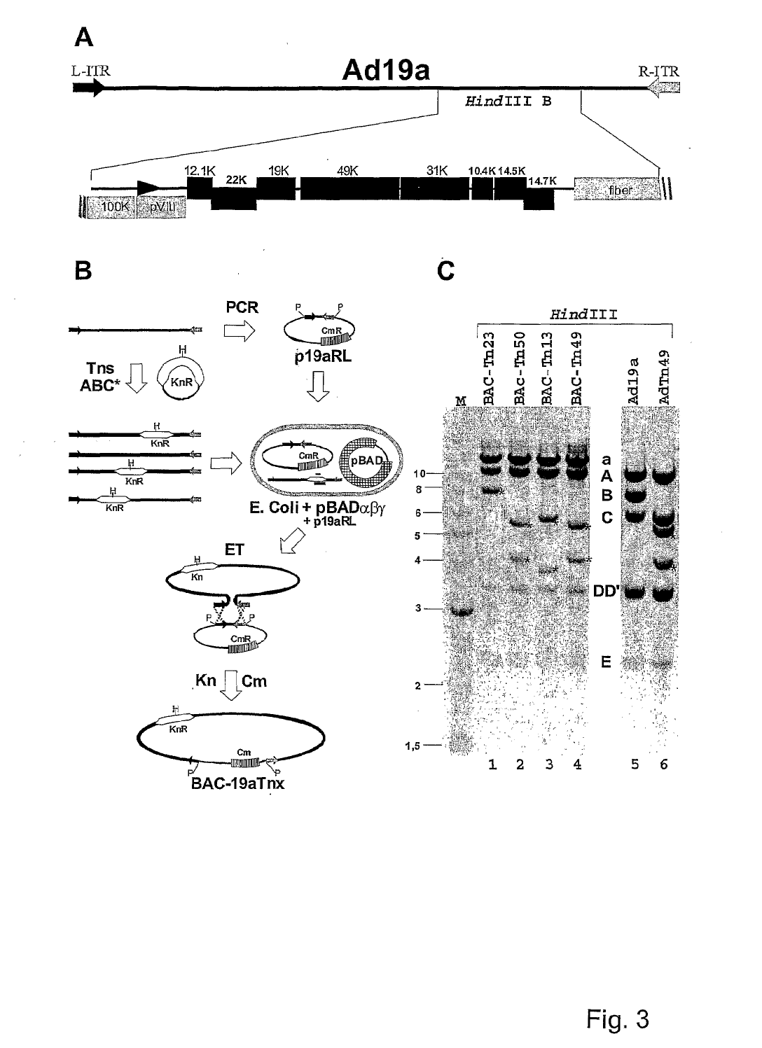

FIG. 3 describes the Transposon-assisted cloning of the Ad19a genome.
(A) Schematic representation of the Ad19a genome. The linear Ad genome is flanked by 135 bp ITRs (black and gray arrows). The HindIII B fragment, comprising the Ad19a E3 region plus flanking sequences on either side, incl. parts of the p100 and fiber ORFs, and pVIII, is shown in detail. The non-essential E3 ORFs (black boxes) and the adjacent essential genes (gray boxes) are indicated.
(B) Schematic representation of the transposon (Tn)-assisted cloning of the Ad19a genome. The PCR-amplified Ad19a ITRs were cloned into the bacterial artificial chromosome (BAC) vector pKSO carrying a chloramphenicol resistance (CmR) gene, thereby generating p19aRL with PacI (P) sites introduced at each ITR-vector border. This entry vector is introduced into electrocompetent E. coli DH10B together with the recombination plasmid pBAD yielding E. coli containing the λ genes αβγ and plasmid p19aRL. In parallel, the purified Ad DNA was labeled by a Tn (white arrows) carrying a kanamycin resistance gene (KnR) and a HindIII site (H) using the TnsABC* in vitro transposase reaction (New England Biolabs, Beverly, USA). ET recombination (ET) was performed by transfecting the Tn-marked Ad DNA into electrocompetent E. coli DH10B containing the p19aRL entry vector and pBAD. Labeled Ad19a genomes containing recombinants were selected by kanamycin (Kn) and chloramphenicol (Cm).
(C) BAC DNA from various colonies was isolated and tested for the presence of Ad hexon sequences by PCR. PacI-treated BAC DNA from hexon-positive clones were transfected into 293 cells. Only BAC DNA with Tn-insertions within the E3 region should yield viable adenoviruses. BAC DNA from selected clones (BAC-Tn23, BAC-Tn50, BAC-Tn13, and BAC-Tn49; lane 1-4) and viral DNA isolated from wt Ad19a (lane 5) and BAC-Tn49-derived reconstituted virus AdTn49 (lane 6) were extracted and digested with HindIII. The typical Ad19a HindIII fragments are indicated (A-E). Fragment A, one of the double fragments DD′, and fragment E is detected in all selected BAC clones. Fragment C and the other DD′ fragment are connected to the vector backbone and form the fragment indicated by ‘a’. The presence of the Tn containing an additional HindIII site in fragment B yields two new bands (‘*’); for Tn23 the second band is not visible in this gel). Ad19aTn49 was reconstituted by transfection of the corresponding PacI-linearised BAC DNA into 293 cells. During virus reconstitution, the plasmid vector is removed, thus, fragment ‘a’ is lost and the normal end fragments (C and one of the DD′ fragments) are revealed (compare lanes 5 and 6). The presence of two new fragments derived from fragment B (*) in the recombinant AdTn49 virus as seen for the parental BAC-Tn49 plasmid indicates that the Tn is maintained during virus replication.

To obtain the Ad19a genome without the Tn, the Tn has to be removed precisely.

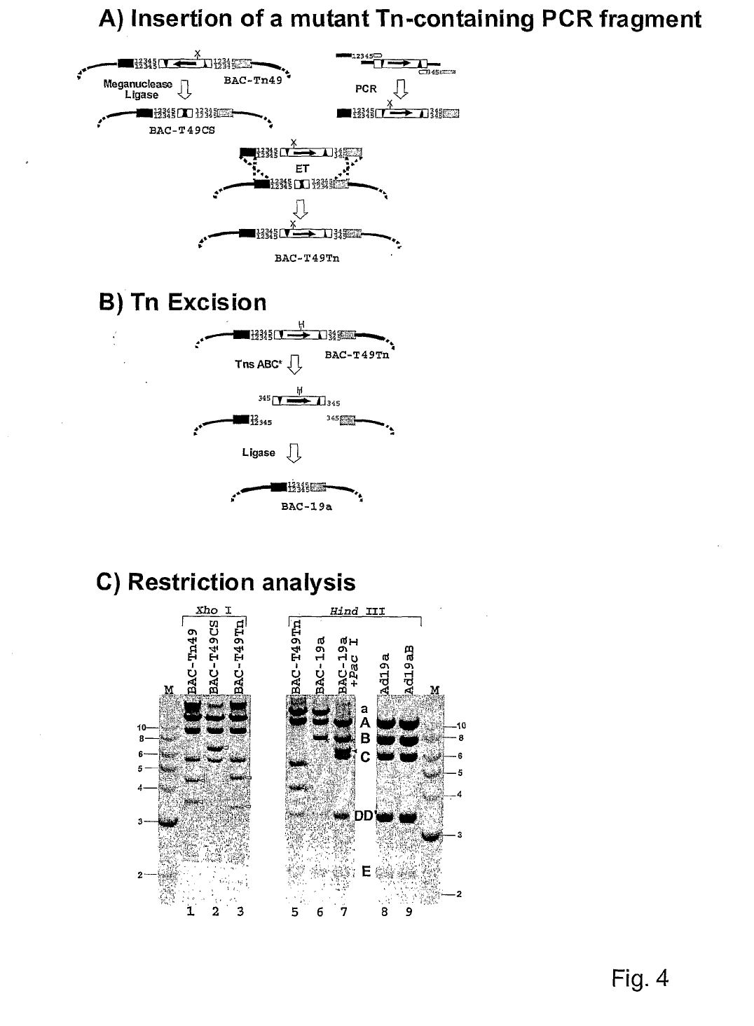

FIG. 4 illustrates the complete removal of the transposon from the Ad19a BAC-Tn49.
(A) Schematic representation of the target repeat mutagenesis. The Kn cassette (arrow) of the Tn (open box) was removed in vitro by I-SceI/I-CeuI meganuclease double digestion (meganuclease sites are indicated by arrowheads) followed by end-filling and ligation generating T49CS. In parallel, a PCR was performed using primers specific to the Tn ends flanked by 40 bp homologies to the target sites in Ad (black and gray boxes). The entire left target repeat was incorporated in the primer whereas only the last 3 bps of the right target repeat were included into the homology region of the right primer. The target repeats are indicated at either side of the Tn by black and gray numbers. This Tn-containing PCR fragment was introduced into the BAC-T49CS by ET recombination. The orientation of the Tn in the newly generated BAC-T49Tn is reversed by the ET cloning step.
(B) Tn-removal from the BAC-T49Tn. The BAC-T49Tn was treated by the TnsABC* transposase complex, which cleaves out the Tn leaving compatible 3 base long 5′ overhangs on the BAC ends. The treated BAC was circularized by simple ligation thereby reconstituting the 5-bp wt Ad target sequence, and thus generating a BAC containing the the wt Ad19a genome (BAC-19a).
(C) Restriction analysis of BAC clones and Ads derived thereof. XhoI pattern of BAC-Tn49, BAC-T49CS and BAC-T49Tn are shown in lanes 1-3, respectively. The Tn-containing fragments are indicated by the white arrowheads. The removal of the Kn cassette from BAC-Tn49 (lane 1) deletes a Tn-encoded XhoI site resulting in only one Tn-containing fragment in BAC-T49CS (lane 2, arrowhead). The re-introduction of the Tn in opposite direction inserts a new XhoI site, thus two Tn-containing fragments are generated after XhoI digestion of BAC-T49Tn (lane 3), which differ in size from those in BAC-Tn49 due to the altered orientation of the Tn. The HindIII pattern of the BAC-T49Tn (lane 5) and BAC-19a (lane 6) shows the removal of the Tn sequences from fragment B after TnsABC* and ligase treatment. The fragment B-derived bands are indicated by ‘*’. The Tn-encoded HindIII site is lost in BAC-19a and a wt-like fragment B appears (lane 6) instead of the two Tn-containing fragments of BAC-T49Tn (compare lane 6 with lane 5). A HindIII-PacI double digest of BAC-19a DNA releases the end fragments (C and one of the DD′ double bands) from the vector backbone (black arrowhead) eliminating fragment ‘a’ (lane 7). The HindIII digest of the DNA purified from BAC-derived virus (Ad19aB; lane 9) is indistinguishable from that of wt Ad19a (Ad19a; lane 8).

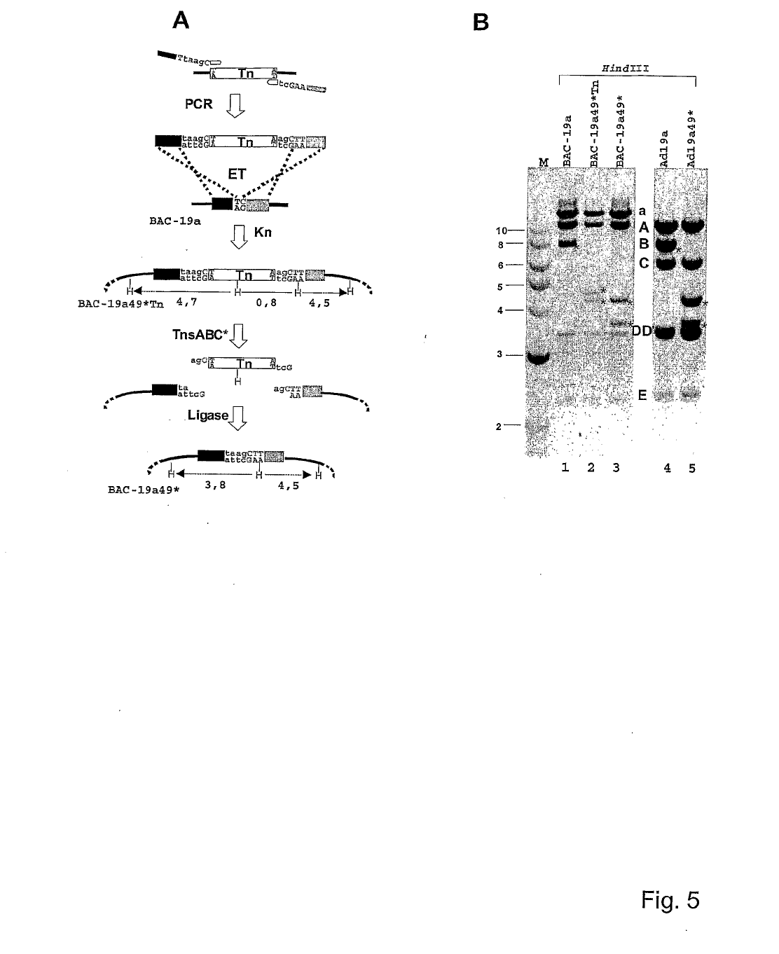

FIGS. 5 and 6 illustrate how modifications of the procedure can be utilized to introduce point mutations, insertions and deletions into the Ad19a genome. We have used this technique to eliminate the E3 and E1 regions of Ad19a and to insert a GFP expression cassette in the E1 region, thereby establishing a replication-deficient Ad19a vector. The steps involved in the so-called “exposon mutagenesis” are shown in FIG. 5A. As an example, a 4-bp insertion was chosen. The mutation (a 4 bp insertion: taag) was introduced into a PCR-amplified recombination fragment. The PCR was performed on a Tn template using ET primers specific for the Tn-ends (22 bp for the left and 30 bp for the right primer) and containing additional 40-base homology arms to the upstream (black boxes) and downstream (gray boxes) target sequences. A 5-base insertion representing the 4 bases of the mutation (small letters) and the first base of the downstream homology (gray capital letters) was placed between the upstream homology arm and the Tn-priming site of the upper primer. In case of the lower primer, the last two bases of the mutation were introduced inbetween the Tn-priming site and the downstream homology arm. After ET recombination (ET) of this fragment and the wt BAC-19a recombinants (BAC-19a49*Tn) were selected by kanamycin (Kn). The purified BAC-19a49*Tn was treated by TnsABC*, which cuts out the Tn and forms compatible 5′ overhangs in the BAC backbone. The TnsABC* treated BAC19a49*Tn was re-circularized by ligation. The resulting mutant BAC19a49* contains only the designed 4 bp insertion and any operational sequence is completely removed.
(B) BAC-19a, BAC-19a49*Tn and BAC-19a49* DNA was extracted and analyzed by HindIII digestion. The bands derived from fragment B are indicated (‘*’). The introduction of the Tn-containing recombination fragment into BAC-19a creates two additional HindIII sites in BAC19a49*Tn (one in the Tn, the other generated by the mutagenesis). This results in three new fragments of 4.7 kb, 4.5 kb, and 0.8 kb (not visible in the figure shown; lane 2) instead of the original fragment B (lane 1). After removal of the Tn sequences only one additional HindIII site (created by the mutagenesis) is left and the 4.7 kb fragment is converted to 3.8 kb (lane 3). Comparison of the HindIII restriction pattern from the reconstituted BAC-derived mutant virus Ad19a49* carrying the mutagenesis-derived HindIII site (lane 5) with that of wt Ad19a (lane 4); On mutagenesis the normal B fragment of Ad19a is converted into the two fragments (*) also seen in the BAC-19a49*.

FIG. 6 illustrates the steps involved when exposon cloning/mutagenesis is utilized for deletion and insertion of genes.
(A) Schematic representation of Tn-assisted deletion (I, left) and insertion of genes (II, right). The Tn-containing PCR-derived recombination fragment was designed such as to contain 40 bp homology arms to the beginning (5′ part) and end (3′ part) of the Ad19a E3 region (black and grey boxes representing the borders of the deletion). After ET recombination with the wt BAC19a the coding region of Ad19a E3 (hatched box) is replaced by the Tn-containing PCR fragment. Three bp direct repeats were introduced by the ET primers at either Tn end (123). The Tn-containing intermediate (BAC-19aΔE3Tn) was cleaved by TnsABC* and circularized via its compatible ends, yielding the deletion mutant BAC-19aΔE3 (I). In a modification of the procedure, a new gene or DNA sequence can be inserted (II). In this case, an Ad19a was generated expressing only one E3 gene, 49K. A PCR-derived 49K protein-encoding insert (checkered box) having compatible sticky ends generated by SapI cleavage was cloned into the TnsABC*-treated BAC-19aΔE3Tn creating BAC-19aΔE3+49K.
(B) BAC DNAs were extracted from the BAC-19a, BAC-19aAE3Tn, BAC-19aΔE3, and BAC-19aΔE3+49K and analyzed by HindIII digestion. The bands derived from fragment B are indicated ‘*’. The E3 coding region in fragment B (lane 1) was replaced by the Tn. Two additional HindIII sites are introduced, one by the mutation and the other by the Tn. Thus, the HindIII digest of the BAC-19aΔE3Tn indicates a big deletion in fragment B (lane 2, *; smaller fragments are not visible). The removal of the Tn from BAC-19aΔE3Tn results in an additional deletion and consequently further migration of the fragment B-derived fragment (lane 3,*; BAC-19aΔE3). The replacement of the Tn of the BAC-19aΔE3Tn by the 49K-coding sequence results in deletion of the Tn and both HindIII sites. Consequently, a larger fragment (6 kb) is visualized carrying the residual fragment B sequences linked to the 49 K-coding region (lane 4; BAC-19aΔE3+49 K). HindIII-digested DNA extracted from wt Ad19a (lane 5) and the reconstituted recombinant viruses Ad19aAE3 (lane 6) and Ad19aAE3+49 K (lane 7) exhibit B-derived fragments with the same size as those seen in the corresponding BAC DNA (compare lanes 5-7 with lanes 1, 3, and 4).

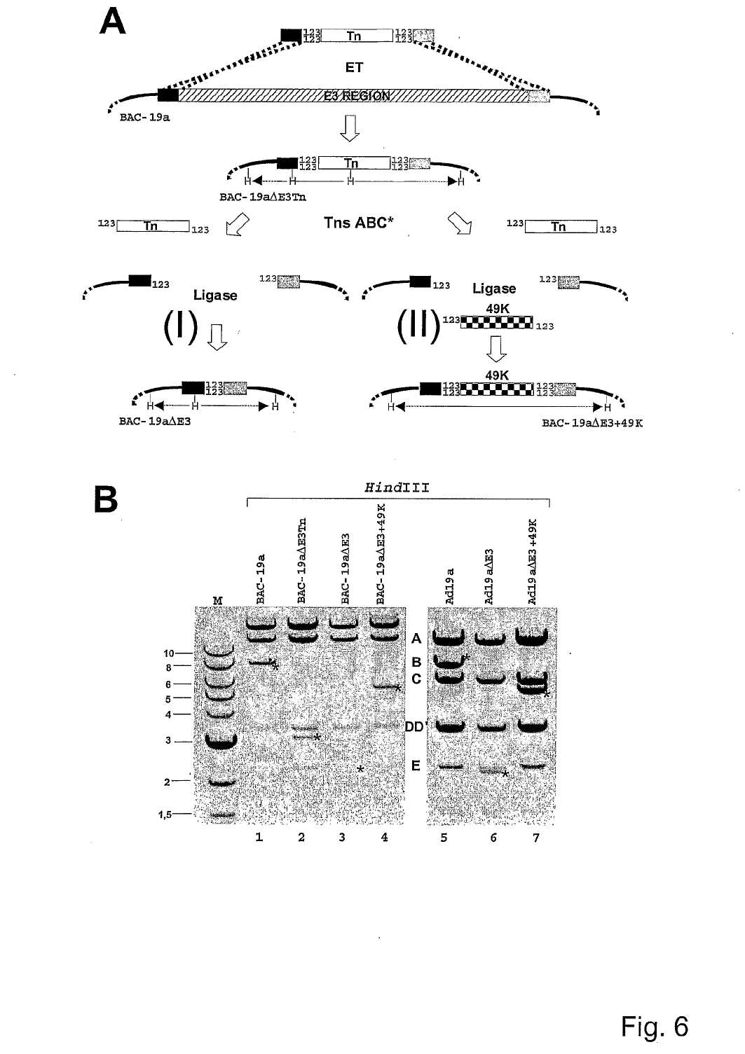

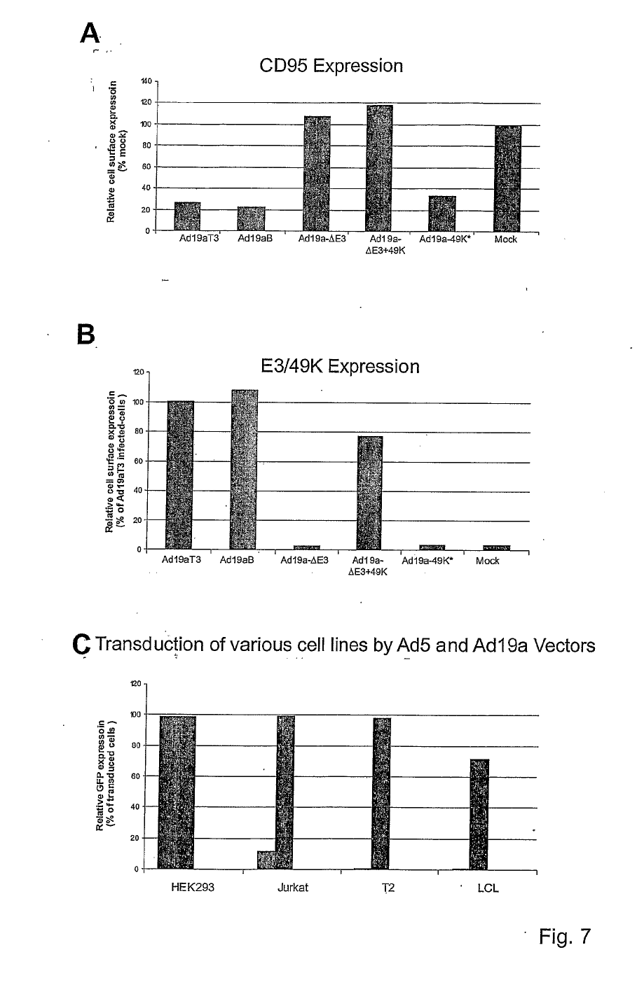

FIG. 7: Phenotypes of various Ad19a mutant viruses generated with the procedure desribed above. (A) Ads remove several apoptosis receptors, including CD95 (Fas), from the cell surface of infected cells to protect them from premature apoptosis. Down-regulation of Fas from the cell surface requires the E3/10.4-14.5 K proteins (also called RID). The capacity of mutant and wt Ad19a to down-regulate CD95 (Fas) was investigated: In wt Ads such as the plaque-purified Ad19a T3 these genes are expressed and, therefore, CD95 is down-regulated as compared to mock-infected cells (100%). A similar down-regulation is observed in BAC-derived wt Ad19a (Ad19aB) or a mutant Ad19a, Ad19a49K*, in which expression of E3/49K (an E3 gene unrelated to Fas down-regulation) is specifically eliminated after insertion of the 4 bp mutation. This demonstrates that elimination of E3/49K (Windheim and Burgert, 2002) expression does not affect the function of E3/RID. In contrast, Ad19a viruses lacking all E3 genes (Ad19aB-AE3) or expressing in the E3 region only E3/49K (Ad19aB-ΔE3+49 K) but not E3/10.4-14.5 are unable to modulate Fas from the cell surface. B) 49 K expression in Ad19a wt and mutant viruses as measured by FACS analysis using a mAb specific for 49 K. Cells infected with plaque-purified wt Ad19a, BAC-derived wt Ad19a (Ad19aB) as well as mutant Ad19a expressing solely 49 K in the E3 region (Ad19aB-ΔE3+49 K) synthesize 49 K whereas those infected with mutant viruses selectively lacking 49 K expression (Ad19a49K*) or lacking all E3 genes (Ad19aB-ΔE3) exhibit only background staining.
C) Comparison of the transduction capacity of GFP-expressing Ad19a and Ad5 vectors. To generate an Ad19a vector expressing GFP, we have deleted the El region of Ad19a by introducing a Tn in this region, using BAC-19aΔE3 as the initial target. In a second round of recombination, we replaced the Tn by an expression cassette encoding green fluorescent protein (GFP) under the control of the CMV immediate early promoter and the SV40 enhancer (see FIG. 8 for the sequence of the insert region). The recombinant E1-negative Ad19a mutant virus was viable in the 293 cell line which expresses the E1 genes of Ad5. The Ad19a-derived recombinant Ad vector, Ad19aΔE1-GFP-ΔE3 was tested for its transducing capacity in different established cell lines and showed a remarkably different transduction pattern as compared to the commonly used Ad5-derived gene therapy vector (FIG. 7). In contrast to the standard Ad5 vector (blue bars), the Ad19a vector efficiently transduced all lymphoid cell lines tested (Jurkat, T2, LCL). In each case, the transduction efficiency was higher than 70%.

### MATERIALS AND METHODS

### Cell Lines, Viruses and Preparation of Viral Genomic DNA

The human epithelial lung carcinoma cell line A549 and the Ad5-transformed human epithelial kidney cell line 293 were cultured in DMEM supplemented with 10% FCS penicillin (100 U/ml) streptomycin (100 μg/ml) and glutamine. The ME strain of human adenovirus type 19a (Wadell and de Jong, 1980); a gift of G. Wadell) was plaque-purified and amplified by infecting subconfluent A549 cells with an moi of 1-2 (1-2 pfu/cell). After the cytopathic effect (CPE) was complete, the infected cells were harvested and washed once in PBS. The cell suspension was lysed by Triton X-100 lysis buffer (1% Triton, 400 mM NaCl, 10 mM Tris pH 7,5). The lysis supernatant was proteinase K-treated in the presence of 0.5% SDS and released virus DNA was treated with RNase A and extracted with phenol-chloroform.

Culture of human Dendritic cells (DCs)

DCs were derived from buffy coats (received from the Red Cross blood bank) using standard methods. Briefly, peripheral blood mononuclear cells were isolated by sedimentation in Ficoll-Hypaque and plated in RPMI supplemented with 5% human serum and antibiotics. After 1 h adsorption, the floating cells were removed and the adherent cells were incubated for 6 or 7 days with GM-CSF (100 IU/ml; Sando) and IL-4 (1000 U/ml). At day 3 and 5 cells were fed with the same amount of cytokines. At day 7, most cells were non-adherent immature DCs (CD14-, CD1+, CD86+). For infection, cells were washed in OptiMEM (Gibco-Invitrogen, Karlsruhe, Germany) and plated in OptiMEM. After 1 h Ad19a (4-10 pfu/cell) was added. After another 1 h, the medium was removed and replaced by RPMI1640, 2-10% inactivated FCS, GM-CSF (100 IU/ml or 50 ng/ml; Sando) and IL-4 (1000 U/ml). In parallel, A549 cells or the primary fibroblasts SeBu (Elsing and Burgert, 1998) were infected with the same amount of virus. Two days later the cells were processed for FACS analysis using various antibodies.

### Flow Cytometry (Fluorescence Activated Cell Sorting; FACS)

Fluorescence activated cell sorting (FACS) was done essentially as described (Elsing and Burgert, 1998; Sester and Burgert, 1994) except that 3-5×10⁵ cells/sample were used. For flow cytotnetty analysis adherent cells (A549 or SeBu) were washed once with PBS and detached with Trypsin/EDTA. DCs were floating or were detached from the plate by vigiorous pipetting. Cells were resuspended in 5 ml DMEM containing 10% FCS, centrifuged (300 g, 5 min) and washed in PBS before they were fixed with formaldehyde (CellFIX, BD Biosciences, Heidelberg, Germany). After quenching with NH₄Cl and further washes in PBS the cells were resuspended in ice-cold FACS buffer (FB; PBS, 2.5% FCS, 0.07% Na azide) supplemented with 0.1% saponin (Sigma, Munich, Germany), FB+SAP, or FB+SAP containing ˜1 μg purified monoclonal antibody 2Hx-2 (ATCC HB-8117) against the hexon. Alternatively, undiluted 2Hx-2 hybridoma supernatant supplemented with 0.1% saponin was used. After incubation for 45 min at 4° C., cells were washed 3 times with FB+SAP, followed by incubation with a FITC-labelled goat anti-mouse secondary antibody (Sigma). After 45 min incubation at 4° C. in the dark, cells were washed three more times with FB+SAP. Fluorescence profiles were obtained by analyzing 5000 viable cells in a FACScalibur flow cytometer using the CellQuest software (BD Biosciences, Heidelberg, Germany). From the mean value of fluorescence, background staining obtained with the secondary Ab alone or an unrelated Ab (e.g. 34-1-2, directed against the murine MHC K^{d} molecule) was deducted.

### Transposon Labeling of the Viral DNA and ET Recombinational Cloning

The BAC entry vector was generated by direct cloning of an assembled PCR product consisting of two Ad19a ITRs connected with a short unique E4 sequence. The ends of the Ad19a genome were amplified by using two different primer pairs. To amplify the “right” end of the genome, primers specific to the terminal virus sequence flanked by a 5′ PacI site and to the conserved E4 sequence close to the right end of the Ad19a genome was used. For generating the “left” ITR fragment, the same terminal primer and a primer specific to the distal ITR sequence flanked by a 15-base homology sequence to the E4 primer were used. The products of the left and the right PCR were combined and re-amplified by the terminal primers. The product of the assembly-PCR was cloned into the PacI site of pKSO BAC vector yielding p19a-RL. pGPS 1.1 (New England Biolabs, Beverly, USA) containing a mini-transposon cassette (Transprimer 1) was used as transposon donor in the transposon-assisted cloning experiment. 200 or 300 ng of purified viral DNA was labeled with the Transprimer-1 in vitro according to the Genome Priming System protocol (New England Biolabs). The recombination-proficient electrocompetent cells were prepared according to a standard protocol using E. coli DH10B (Invitrogen, Karlsruhe, Germany) transformed by the pl9a-RL and pBADαβγ induced with 0,1% L-arabinose (Muyrers et al., 2000; Zhang et al., 1998). The recombination-proficient electrocompetent cells were transformed with labeled Ad19a DNA using the BioRad. GenePulser Apparatus with the following settings: 2500 V, 200Ω and 25 μF. The transformants were plated on LB agar plates containing 25μg/ml of chloramphenicol and 20 μg/ml kanamycin. The isolated colonies were screened by PCR after boiling using primers specific to the Ad hexon.

### ET Recombination for Mutagenesis

Synthetic oligonucleotide ET primers consisting of the up- and downstream homology arms, different insertion sequences and priming sequences located exactly at the left and right end of the Transprimer-1 casettes of pGPS1.1 were used. The sequences of the priming regions (representing the 3′ ends of the primers) were always 5′-TGT GGG CGG ACA AAA TAG TTG G -3′ (specific to the left end) and 5′-TGT GGG CGG ACA ATA AAG TCT TAA ACT GAA-3′ for the right end of Transprimer-1. Specific 40nt homology regions were added at the 5′ end of the ET primers as needed. The 3-nucleotide direct repeats were included between the homology arms and the priming regions of each primers. PCRs were done using the Expand High Fidelity PCR System (Roche Diagnostics, Mannheim, Germany) and PCR products were purified with the PCR purification kit (Qiagen, Hilden, Germany). E. coli DH10 B cells were co-transformed with the target BACs and pBAD-αβγ and electrocompetent cell were prepared. For ET recombination (Muyrers et al., 2000) 0.3-0.4 μg of the purified recombination fragment was transformed into the induced target cells by electroporation, as described above. After 1.5 hour growth in 1 ml LB at 37° C. the transformants were plated on LB agar plates containing 25 μg/ml chloramphenicol and 20 μg/ml kanamycin.

### Transposon Excisions

140 ng of the Tn-containing BACs were treated with 1 μTnsABC* (New England Biolabs, Beverly, USA) in the presence of 90 ng of temperature-sensitive plasmid pST76T as dead-end target in lx GPS buffer (New England Biolabs, Beverly, USA). After 10 min incubation at 37 ° C. 1/20 vol. of 0.3 M MgCl₂ was added to initiate Tn end cleavage. The reactions were stopped after 60 min incubation at 37° C. by heat treatment. 400 cohesive end units of T4 ligase were added and the reaction mixture incubated for re-circularization at 16° C. overnight. After heat inactivation of the T4 ligase the reaction mixtures were phenol-chloroform extracted and ethanol preciptated. Electrocompetent E. coli DH10B or I-SceI expressing pUC19RP12-transformed E. coli DH10B were transformed with the purified reaction products. In the exposon cloning reaction, 200 ng of purified SapI treated inserts were added to the heat inactivated TnsABC* reaction prior to T4 ligase treatment. Transformants were plated on LB agar plates containing 25μg/ml of chloramphenicol.

### Reconstitution of the Recombinant Viruses

Recombinant viruses were reconstituted by transfection of 50% confluent 293 cells in 6 cm² cell culture dishes by a standard Ca-phosphate precipitation method using PacI-linearized Ad19a-BACs. Transfected cells were incubated with the transfection mixtures overnight and were split into 10 cm dishes 48 h post-transfection. After development of a complete CPE, the recombinant virus stocks were prepared by standard protocols.

### Sequencing of the Ad19a Genome

Sequencing of the Ad19a genome was carried out in several steps. First, the Ad19a E3 region was sequenced (Blusch et al., 2002; Burgert and Blusch, 2000; Deryckere and Burgert, 1996). Subsequently, a partial hexon sequence (AF271989) as well as that of the ITRs (AF271991) was obtained. Fiber and hexon were previously sequenced (Arnberg et al., 1997; Crawford-Miksza and Schnurr, 1996). The left end of the genome, the Ad19aE1 region including the pIX gene (4010 bp) and the right end, the E4 region was sequenced by using the GPS-1 Genome priming system (New England Biolabs, Beverly, USA) according to the manufacturer's instructions. The transposon from the pGPS 1.1 Transprimer donor plasmid was randomly inserted in vitro into the target DNAs. Clones were isolated, roughly mapped by restriction enzymes and the Ad sequences flanking the transposon determined using the right and left transposon primer. The missing sequence between the hexon and the E3 region (8300 bp), the hexon and pIX and the fiber and E4 was established following a primer-walking strategy. Unless otherwise stated, direct dideoxy terminator cycle sequencing was performed for both strands using ABI 373A or 377 DNA sequencers (Applied Biosystems, Weiterstadt, Germany). For assembly of the sequenced fragments the Lasergene SeqManII software (DNASTAR Inc., version 3.14) was used.

### Reference List

Arnberg, N., Edlund, K., Kidd, A. H., and Wadell, G. (2000). Adenovirus type 37 uses sialic acid as a cellular receptor. J Virol 74, 42-48.
Arnberg, N., Mei, Y., and Wadell, G. (1997). Fiber genes of adenoviruses with tropism for the eye and the genital tract. Virology 227, 239-244.
Balkwill, F. (2004). Cancer and the Chemokine network. Nat Rev Cancer 4, 540-550.
Banchereau, J., Briere, F., Caux, C., Davoust, J., Lebecque, S., Liu, Y. J., Pulendran, B., and Palucka, K. (2000). Immunobiology of dendritic cells. Annual Review of Immunology 18, 767-811.
Bergelson, J. M., Cunningham, J. A., Droguett, G., Kurt-Jones, E. A., Krithivas, A., Hong, J. S., Horwitz, M. S., Crowell, R. L., and Finberg, R. W. (1997). Isolation of a common receptor for Coxsackie B viruses and adenoviruses 2 and 5. Science 275, 1320-1323.
Blusch, S. H., Deryckere, F., Windheim, M., Ruzsics, Z., Arnberg, N., Adrian, T., and Burgert, H. G. (2002). The Novel Early Region 3 Protein E3/49K Is Specifically Expressed by Adenoviruses of Subgenus D: Implications for Epidemic Keratoconjunctivitis and Adenovirus Evolution. Virology 296, 94-106.
Brummelkamp, T. R., Bernards, R., and Agami, R. (2002). A System for Stable Expression of Short Interfering RNAs in Mammalian Cells. Science 296, 550-553.
Burgert, H. G., and Blusch, J. H. (2000). Immunomodulatory functions encoded by the E3 transcription unit of adenoviruses. Virus Genes 21, 13-25.
Crawford-Miksza, L. K., and Schnurr, D. P. (1996). Adenovirus serotype evolution is driven by illegitimate recombination in the hypervariable regions of the hexon protein. Virology 224, 357-367.
De Jong, J. C., Wermenbol, A. G., Verweij-Uijterwaal, M. W., Slaterus, K. W., Wertheim-Van Dillen, P., Van Doornum, G. J., Khoo, S. H., and Hierholzer, J. C. (1999). Adenoviruses from human immunodeficiency virus-infected individuals, including two strains that represent new candidate serotypes Ad50 and Ad51 of species B1 and D, respectively. J Clin Microbiol 37, 3940-3945.
Deryckere, F., and Burgert, H. G. (1996). Early region 3 of adenovirus type 19 (subgroup D) encodes an HLA-binding protein distinct from that of subgroups B and C. J Virol 70, 2832-2841.
Dobbelstein, M. (2004). Replicating adenoviruses in cancer therapy. Curr Top Microbiol Immunol 273, 291-334.
Doronin, K., Toth, K., Kuppuswamy, M., Ward, P., Tollefson, A. E., and Wold, W. S. (2000). Tumor-specific, replication-competent adenovirus vectors overexpressing the adenovirus death protein. Journal of Virology 74, 6147-6155.
Dykxhoorn D M, N. C., Sharp PA. (2003). Killing the messenger: short RNAs that silence gene expression. Nat Rev Mol Cell Biol 4, 457-467.
Elsing, A., and Burgert, H.-G. (1998). The adenovirus E3/10.4K-14.5K proteins down-modulate the apoptosis receptor Fas/Apo-1 by inducing its internalization. Proc Natl Acad Sci USA 95, 10072-10077.
Horwitz, M. S. (2001). Adenoviruses. In Fields Virology, D. M. Knipe, and P. M. Howley, eds. (Lippincoft Williams & Wilkins), pp. 2301-2326.
Imperiale, M.; and Kochanek, S. (2004). Adenovirus vectors: biology, design, and production. Curr Top Microbiol Inununol 2 73, 335-357.
Kanerva, A., and Hemminki, A. (2004). Modified adenoviruses for cancer gene therapy. Int J Cancer 110., 475-480.
Kerbel, R., and Folkman, J. (2002). Clinical translation of angiogenesis inhibitors. Nature Rev Cancer 2, 727-739 (2002); 2, 727-739.
Kim, M., Zinn, K. R., Barnett, B. G., Sumerel, L. A., Krasnykh, V., Curiel, D. T., and
Douglas, J. T. (2002). The therapeutic efficacy of adenoviral vectors for cancer gene therapy is limited by a low level of primary adenovirus receptors on tumour cells. Eur J Cancer 38, 1917-1926.
Mantovani, A., Allavena, P., Sozzani, S., Vecchi, A., Locati, M., and Sica, A. (2004). Chemolcines in the recruitment and shaping of the leukocyte infiltrate of tumors. Seminars in Cancer Biology 14, 155-160.
Muyrers, J. P., Zhang, Y., Benes, V., Testa, G., Ansorge, W., and Stewart, A. F. (2000). Point mutation of bacterial artificial chromosomes by ET recombination. EMBO Rep 1, 239-243.
Nemerow, G. R. (2000). Cell receptors involved in adenovirus entry. Virology 274, 1-4.
Palmer, D., Mautner, V., and Kerr, D. (2002). Clinical experience with adenovirus in cancer therapy. Curr Opin Mol Ther 4, 423-434.
Roelvink, P. W., Lizonova, A., Lee, J. G., Li, Y., Bergelson, J. M., Finberg, R. W., Brough, D. E., Kovesdi, I., and Wickham, T. J. (1998). The coxsackievirus-adenovirus receptor protein can function as a cellular attachment protein for adenovirus serotypes from subgroups A, C, D, E, and F. J Virol 72, 7909-7915.
Ross R, S. S., Beisner J, Ross XL, Ludwig-Portugall I, Steitz J, Tuting T, Knop J, Reske-Kunz AB. (2003). Transcriptional targeting of dendritic cells for gene therapy using the promoter of the cytoskeletal protein fascin. Gene Ther 10, 1035-1040.
Rowe, W. P., Huebner, R. J., Gilmore, L. K., Rarrot, R. H., and Ward, T. G. (1953). Isolation of a cytopathogenic agent from human adenoids undergoing spontaneous degeneration in tissue culture. Proc Soc Exp Biol Med 84, 570-573.
Russell, W. C. (2000). Update on adenovirus and its vectors. J Gen Virol 81, 2573-2604.
Sambroolc, J., Fritsch, E. F., and Maniatis, T. (1989). Molecular Cloning: A Laboratory Manual, 2nd ed., Cold Spring Harbor Laboratory Press, Cold Spring Harbor, N.Y.).
Schuler, G., Schuler_Thumer, B., and Steinman, R. M. (2003). The use of dendritic cells in cancer immunotherapy. Curr Opin Immunol 15, 138-147.
Sester, M., and Burgert, H.-G. (1994). Conserved cysteine residues within the E3/19K protein of adenovirus type 2 are essential for binding to major histocompatibility complex antigens. J Virol 68, 5423-5432.
Shenk, T. E. (2001). Adenoviridae: The viruses and their replication. In Fields Virology, D. M. Knipe, and P. M. Howley, eds. (Lippincott Williams & Wilkins), pp. 2265-2300.
St George, J. (2003). Gene therapy progress and prospects: adenoviral vectors. Gene Ther 10, 1135-1141.
van der Bruggen, P., Zhang, Y., Chaux, P., Stroobant, V., Panichelli, C., Schultz, E. S., Chapiro, J., Van den Eynde, B. J., Brasseur, F., and Boon, T. (2002). Tumor-specific shared antigenic peptides recognized by human T cells. Immunol Rev 188, 51-64.
Volpers, C., and Kochanek, S. (2004). Adenoviral vectors for gene transfer and therapy. J Gene Med 6, S164-S171.
Wadell, G., and de Jong, J. C. (1980). Restriction endonucleases in identification of a genome type of adenovirus 19 associated with keratoconjunctivitis. Infect Immun 27, 292-296.
Windheim, M., and Burgert, H. G. (2002). Characterization of E3/49K, a novel, highly glycosylated E3 protein of the epidemic keratoconjunctivitis-causing adenovirus type 19a. J Virol 76, 755-766.
Wu, E., Fernandez, J., Fleck, S. K., Von Seggern, D. J., Huang, S., and Nemerow, G. R. (2001). A 50-kDa membrane protein mediates sialic acid-independent binding and infection of conjunctival cells by adenovirus type 37. Virology 279, 78-89.
Zhang, Y., Buchholz, F., Muyrers, J. P., and Stewart, A. F. (1998). A new logic for DNA engineering using recombination in Escherichia coli. Nat Genet 20, 123-128.

1. An isolated adenovirus, wherein the genome of said adenovirus comprises the nucleic acid sequence shown in FIG. 2, or a variant adenovirus wherein said variant adenovirus genome is modified by the addition, deletion or substitution of at least one nucleotide base and hybridises under stringent hybridization conditions to the nucleic acid molecule shown in FIG. 2.

2. An adenovirus according to claim 1, wherein said adenovirus genome is modified by the inclusion of at least one heterologous nucleic acid molecule.

3. An adenovirus according to claim 2, wherein said genome is adapted for eukaryotic expression of said heterologous nucleic acid molecule.

4. An adenovirus according to claim 2, wherein the expression of said nucleic acid molecule is controlled by a cell-specific promoter.

5. An adenovirus according to claim 4, wherein said cell-specific promoter is a cancer cell specific promoter.

6. An adenovirus according to claim 2, wherein said heterologous nucleic acid molecule encodes a therapeutic agent.

7. An adenovirus according to claim 6, wherein said therapeutic agent is a polypeptide.

8. An adenovirus according to claim 7, wherein said polypeptide is selected from the group consisting of an antigenic polypeptide, a cytotoxic agent, a polypeptide that induces cell-cycle arrest, a pharmaceutically active polypeptide, a cytokine, a chemokine, an antibody, an active binding fragment of an antibody, a tumor suppressor polypeptide, a pro-apoptotic factor, p53, a polypeptide that induces cell death as opposed to apoptosis, a prodrug-activating polypeptide and a peptide having anti-angiogenic activity.

9. An adenovirus according to claim 8, wherein said antigenic polypeptide is a tumor antigen, or contains part of at least one tumor antigen.

10. (canceled)

11. An adenovirus according to claim 8, wherein said antigenic polypeptide is an antigen of an infectious agent.

12. (canceled)

13. An adenovirus according to claim 8, wherein said polypeptide is a cytotoxic agent selected from the group consisting of pseudomonas exotoxin, ricin toxin and diphtheria toxin.

14-18. (canceled)

19. An adenovirus according to claim 8, wherein said polypeptide is an active binding fragment of an antibody comprising a Fab fragment or a single chain antibody variable fragment.

20-26. (canceled)

27. An adenovirus according to claim 2, wherein said heterologous nucleic acid molecule encodes an antisense nucleic acid molecule, an interfering ribonucleic acid molecule (RNAi) or a small interfering RNA (siRNA) or a short hairpin RNA (shRNA).

28. An adenovirus according to claim 1, wherein said adenovirus is replication-deficient.

29. An adenovirus according to claim 28 wherein said adenovirus has a modified E1 region that renders said virus replication-deficient or conditionally replication-competent.

30. (canceled)

31. An adenovirus according to claim 29, wherein said adenovirus has a modified E1 and a modified E3 region.

32. An adenovirus according to claim 2 wherein said adenovirus further comprises a heterologous nucleic acid that encodes a proteinaceous fluorophore.

33-39. (canceled)

40. An isolated cell comprising the adenovirus according to claim 1, wherein said cell expresses low levels, or does not express detectable levels, of coxsackie adenovirus receptor.

41-44. (canceled)

45. An isolated cell comprising the adenovirus of claim 1, wherein said cell is an ocular cell, a corneal cell, a conjunctive cell or a retinal cell.

46-66. (canceled)

67. A pharmaceutical composition comprising the adenovirus according to claim 1.

68. (canceled)

69. A method of treating cancer in an animal or human comprising administering a therapeutically effective amount of the adenovirus according to claim 1 to said animal or human.

70. (canceled)

71. (canceled)

72. An adenovirus according to claim 1, wherein said adenovirus is a high capacity adenovirus vector.

73. A chimeric adenovirus comprising a first nucleic acid comprising an adenovirus nucleic acid, or part thereof according to claim 1, and at least one second nucleic acid comprising an adenovirus nucleic acid derived from a different Ad serotype.

74. A chimeric adenovirus comprising a first nucleic acid encoding an adenovirus 19a fiber or modified fiber polypeptide or part thereof and at least one second nucleic acid comprising an adenovirus nucleic acid not derived from a Ad19a serotype.

75. A chimeric adenovirus according to claim 74, wherein said second nucleic acid is derived from Ad2 or AdS.

76-100. (canceled)

101. A pharmaceutical composition comprising the cell according to claim 40.

102. A method of infecting a cell with an adenovirus, wherein said cell expresses low levels, or does not express detectable levels, of coxsackie adenovirus receptor, comprising exposing said cell to an adenovirus comprising a fiber of a subgenus D adenovirus, wherein said virus is capable of infecting said cell.

103. The method according to claim 102, wherein said virus comprises a fiber of Ad8, Ad19a or Ad37 or parts thereof.

104. The method according to claim 102, wherein said adenovirus is an adenovirus of subgenus D.

105. The method according to claim 102, wherein the adenovirus is Ad8, Ad 19a or Ad 37.

106. The method according to claim 102, wherein the subgenus D adenovirus is Ad19a.

107. The method according to claim 102, further comprising inhibition of expression of a cellular gene, said method comprising introducing an isolated adenovirus into a cell, wherein the genome of said adenovirus comprises the nucleic acid sequence shown in FIG. 2, or a variant adenovirus wherein said variant adenovirus genome is modified by the addition, deletion or substitution of at least one nucleotide base and hybridizes under stringent hybridization conditions to the nucleic acid molecule shown in FIG. 2, wherein said adenovirus is modified by the inclusion of at least one heterologous nucleic acid molecule which encodes an interfering ribonucleic acid molecule (RNAi) or a small interfering RNA (siRNA) or a short hairpin RNA (shRNA) that hybridizes to mRNA transcribed from said cellular gene.

108. The method according to claim 102, wherein said cell is selected from the group consisting of an ocular cell, a lung cell, a hematopoietic cell, an endothelial cell, a muscle cell, a neuron and a cancer cell.

109. The method according to claim 108, wherein said ocular cell is a corneal cell, a conjunctival cell or a retinal cell.

110. The method according to claim 108, wherein said lung cell is a differentiated lung epithelial cell or bronchial epithelial cell.

111. The method according to claim 108, wherein said hematopoietic cell is a hematopoietic stem cell, leukocyte or lymphocyte.

112. A method according to claim 108, wherein said muscle cell is a cardiac muscle cell, a striated muscle cell or a smooth muscle cell.

113. The method according to claim 108, wherein said cancer cell is selected from the group consisting of a lymphoid cancer cell, a glioma cell, an androgen resistant prostate cancer cell, a melanoma cell, a bladder cancer cell, an ovarian cancer cell, a colorectal cancer cell and a cervical cancer cell.

114. The method of transducing a cell with a gene of interest, wherein said cell expresses low levels, or does not express detectable levels, of cell surface coxsackie adenovirus receptor, comprising exposing said cell to an adenovirus comprising a fiber of a subgenus D adenovirus, or part thereof, such that said virus is capable of infecting said cell, wherein said adenovirus comprises the gene of interest.

115. The method according to claim 114, wherein said virus comprises a fiber of Ad8, Ad19a or Ad37 or part thereof.

116. The method according to claim 114, wherein said adenovirus is an adenovirus of subgenus D.

117. The method according to claim 114, wherein the adenovirus is Ad8, Ad19a or Ad 37.

118. The method according to claim 114, wherein the subgenus D adenovirus is Ad19a.

119. The method according to claim 114 wherein said cell is an antigen-presenting cell

120. The method according to claim 119 wherein said antigen-presenting cell is a dendritic cell.

121. A method of transducing a cell and expressing a gene of interest, comprising exposing said cell to an adenovirus comprising a fiber of an adenovirus of subgenus D, or part thereof, wherein said virus is capable of infecting said cell, and wherein said adenovirus comprises the gene of interest.

122. The adenovirus according to claim 121, wherein said adenovirus is an adenovirus that causes epidemic keratoconjunctivitis.

123. The adenovirus according to claim 122 wherein said subgenus D adenovirus is selected from the group consisting of Ad8, 9, 10, 13, 15, 17, 19a, 19p, 20, 22, 23, 24, 25, 26, 27, 28, 29, 30, 32, 33, 36, 37, 38, 39, 42, 43, 44, 45, 46, 47, 48, 49 and 51.

124. The method according to claim 123, wherein the subgenus D adenovirus is Ad19a.

125. A method of vaccinating an animal or human against an infectious agent or a tumor antigen, comprising transducing a dendritic cell of said animal or human by exposure to an effective amount of an adenovirus comprising a fiber of an adenovirus of subgenus D, or part thereof.

126. The method according to claim 125, wherein said adenovirus comprises a fiber of Ad19a.

127. The method according to claim 125, wherein said adenovirus is Ad19a.


SEQUENCE LISTING (3 sequences)

SEQ ID NO: 1 - DNA, 35216 bp, Adenovirus type 19a
ctatctaataatataccccacaaagtaaacaaaagttaatatgcaaatgagcttttgaattttaacggttttggggcggagccaacgctgattggacgagaagcggtgatgcaaataacgtcacgacgcacggctaacggccggcgcggaggcgtggcctaggccggaagcaagtcgcggggctaatgacgtataaaaaagcggactttagacccggaaacggccgattttcccgcggccacgcccggatatgaggtaattctgggcggatgcaagtgaaattaggtcattttggcgccaaaactgaatgaggaagtgaaaagtgaaaaatacctgtcccgcccagggcggaatatttaccgagggccgagagactttgaccgattacgtggggtttcgattgcggtgtttttttcgcgaatttccgcgtccgtgtgaaagtccggtgtttatgtcacagatcagctgatccacagggtatttaaaccagttgagcccgtcaagaggccactcttgagtgccagcgagtagagatttctctgagctccgctcccaaagtgtgagaaaaatgagacacctgcgcctcctgtcttcaactgtgcctattaacatggccgcattattgctggaggactatgtgagtacagtattggaggacgaactacatccatctccatttgagctgggacctacacttcaggacctttatgatttggaggtagatgcccatgatgacgacccaaacgaagaggctgtgaatttaatatttccagaatctctgattcttcaggctgacatagccagcgaagctgtacctacaccacttcatacaccgactttgtcacccatacctgaattggaagaggaggacgagttagacctccgatgttatgaggaaggttttcctcccagcgattcagaggacgaacagggtgagcagagcatggctctaatctcagaatatgcttgtgtggttgtggaagagcattttgtgttggacaatcctgaggtgcccgggcaaggctgtagatcctgccagtaccaccgggataagaccggagacacaaacgcctcctgcgctctgtgttacatgaaaaagaacttcagctttatttacagtaagtggagtgaatgtgagagaggctgagtgcttaacacataactgggtgatgcttaaacagctgtgctaagtgtggtttatttttgtttctaggtccggtgtcagaggatgagtcatcaccctcagaagaaaaccacccgtgtccccctgagctgtcaggcgaaacgcccctgcaagtgcacaaacccaccccagtcagacccagtggcgagaggcgagcagctgttgaaaaaattgaggacttgttacatgacatgggtggggatgaacctttggacctgagcttgaaacgccccaggaactaggcgcagctgtgcttagtcatgtgtaaataaagttgtacaataaaagtatatgtgacgcatgcaaggtgtggtttatgactcatgggcggggcttagtcctatataagtggcaacacctgggcactgggcacagaccttcagggagttcctgatggatgtgtggactatccttgcagactttagcaagacacgccggcttgtagaggatagttcagacgggtgctccgggttctggagacactggtttggaactcctctatctcgactggtgtacacagttaagaaggattataacgaggaatttgaaaatctttttgctgattgctctggcctgctagattctctgaatctcggccaccagtcccttttccaggaaagggtactccacagccttgatttttccagcccagggcgcactacagccggggttgcttttgtggtttttctggttgacaaatggagccagaacacccaactgagcaggggctacattctggacttcgcagccatgcacctgtggagggcatgggtgaggcagcggggacagagaatcttgaactactggcttatacagccagcagctccgggtcttcttcgtctacacagacaaacatccatgttggaggaagaaatgaggcaggccatggacgagaacccgaggagcggcctggaccctccgtcggaagaggagctggattgaatcaggtatccagcttgtacccagagcttagcaaggtgctgacatccatggctaggggagtgaagagggagaggagcgatgggggcaataccgggatgatgaccgagctgacggccagcctgatgaatcgcaagcgcccagagcgcattacctggcacgagctacagatggagtgcagggatgagttgggcctgatgcaggataaatatggcctggagcagataaaaacacattggttgaacccagatgaggattgggaggaggccattaagaaatatgccaagatagccctgcgcccagattgcaagtacatagtgaccaagaccgtgaatattagacatgcctgctacatttcagggaacggggcagaggtggtcatcgataccctggacaaggccgccttcaggtgttgcatgatgggaatgagagcaggagtgatgaatatgaattccatgatcttcatgaacatgaagttcaatggagagaagtttaatggggtgctgttcatggccaacagccacatgaccctgcatggctgcagtttctttggcttcaacaatatgtgcgccgaggtctggggcgcttccaagatcaggggatgtaagttttatggctgctggatgggcgtggtcggaagacctaagagcgagatgtctgtgaagcagtgtgtgtttgagaaatgctacctgggagtctctaccgagggcaatgctagagtgagacactgctcttccctggatacgggctgcttctgcctggtgaagggtacggcctctctgaagcataatatggtgaagggctgcacagatgagcgcatgtacaacatgctaacatgcgactcgggggtctgtcatatcctgaagaacatccatgtgacctcccaccccagaaagaagtggccagtgtttgagaataacctgctgatcaagtgccatatgcacctgggtgccagaaggggcaccttccagccgtaccagtgcaactttagccagaccaagctgctgttggaaaacgatgccttctccagggtgaacctgaacggcatctttgacatggatgtctcggtgtacaagatcctgagatacgatgagaccaagtccagggtgcgcgcttgcgagtgcgggggcagacacaccaggatgcagccagtggccctggatgtgaccgaggagctgagaccagaccacctggtgatggcctgtaccgggaccgagttcagctccagtggggaggacacagattagaggtaggtttgagtagtgggcgtggctaatgtgagtataaaggcgggtgtcttacgagggtctttttgcttttctgcagacatcatgaacgggaccggcggggccttcgaaggggggctttttagcccttatttgacaacccgcctgccgggatgggccggagttcgtcagaatgtgatgggatctacggtggatgggcgtccagtgcttccagcaaattcctcgaccatgacctacgcgaccgtggggagctcgtcgcttgacagcaccgccgcagccgcggcagccgcagccgccatgacagcgacgagactggcctcgagctatatgcccagcagcggtagcagcccctctgtgcccagttccatcatcgccgaggagaaactgctggccctgctggccgagctggaagccctgagccgccagctggccgccctgacccagcaggtgtccgatctccgcgagcaacagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgggggtggaggtagcaccactgcatggcctcgtgctctggggtcgtgttgtagataatccagtcatagcaggggcgctgggcgtggtgctggatgatgtccttgaggaggagactgatggccacggggagccccttggtgtaggtgttggcaaagcggttaagctgggagggatgcatgcggggggagatgatgtgcagtttggcctggatcttgaggttggcgatgttgccacccagatcccgccgggggttcatattgtgcaggaccaccagaacggtgtagcccgtgcacttggggaacttatcatgcaacttggaagggaatgcgtggaagaatttggagacgcccttgtgcccgcccaggttttccatgcactcatccatgatgatggcaatgggcccgtgggctgcggctttggcaaaaacgtttctggggtcagagacatcataattatgctcctgggtgagatcatcataagacattttaatgaatttggggcgaagggtgccagattgggggacgatcgttccctcgggccccggggcgaagttcccctcgcagatctgcatctcccaggctttcatctcggagggggggatcatgtccacctgcggggcgatgaaaaaaacggtttccggggcgggggtgatgagctgcgaggagagcaggtttcttaacagctgggacttgccgcacccggtcgggccgtagatgaccccgatgacgggttgcaggtggtagttcaaggagatgcagctgccgtcgtcccggaggaggggggccacctcgttgagcatgtctctcacttggaggttttcccggacgagctcgccgaggaggcggtccccgcccagcgagagcagctcttgcagggaagcaaagtttttcaggggcttgagcccgtcggccatgggcatcttggcaagggtctgcgagaggagctccaggcggtcccatagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgagtgagggtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagcttgtagttaagggcctcggcggcgtggcccttggcacggagcttgcctttggaagagcgcccgcaggcgggacagaggagggattgcagggcgtagagcttgggtgcgagaaagacggactcgggagcgaaggcgtccgctccgcagtgggcgcagacggtctcgcactcgacgagccaggtgagctcgggctgctcggggtcaaaaaccagttttcccccgttctttttgatgcgcttcttacctcgcgtctccatgagtctgtgtccgcgttcggtgacaaacaggctgtctgtgtccccgtagacggacttgattggcctgtcctgcaggggcgtcccgcggtcctcctcgtagagaaactcggaccactctgagacaaaggcgcgcgtccacgccaagacaaaggaggccacgtgcgaggggtagcggtcgttgtccaccagggggtccaccttttccaccgtgtgcagacacatgtccccctcctccgcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctcgagagcgggcatgacctcggcactcaggttgtcagtttctagaaacgaggaggatttgatgttggcctgccctgccgcaatgctttttaggagactttcatccatctggtcagaaaagactatttttttattgtcaagcttggtggcaaaggagccatagagggcgttggagagaagcttggcgatggatctcatggtctgatttttgtcacggtcggcgcgctccttggccgcgatgttgagctggacatactcgcgcgcgacacacttccattctgggaagacggtggtgcgctcgtcgggcacgatcctgacgcgccagccgcgattatgcagggtgaccaggtccacgctggtggccacctcgccgcgcaggggctcgttggtccagcagaggcgtccgcccttgcgcgagcagaacgggggcagcacatcaagcagatgctcgtcaggggggtccgcatcgatggtgaagatgcccggacagagttccttgtcaaaataatcgatttttgaggatgcatcatccaaggccatctgccactcgcgggcggccagcgctcgctcgtaggggttgaggggcggaccccagggcatgggatgcgtgagggcggaggcgtacatgccgcagatgtcgtagacatagatgggctccgagaggatgccgatgtaggtgggataacagcgccccccgcggatgctggcgcgcacatagtcatacaactcgtgcgagggggccaagaaagcggggccgagattggtgcgctggggctgctcggcgcggaagacgatctggcgaaagatggcatgcgagttggaggagatggtgggccgttggaagatgttaaagtgggcgtggggcaagcggaccgagtcgcggatgaagtgcgcgtaggagtcttgcagcttggcaacgagctcggcggtgacaaggacgtccatggcgcagtagtccagcgtttcacggatgatgtcataacccgcctcttctttcttctcccacagcgcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtatccctaaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccggaacggaggctcggttgttgatgacctgggcggcgaggacgatctcgtcgaagccgttgatgttgtgcccgacgatgtagagttccatgaatcgcgggcggcctttgatgtgcggcagctttttgagttcctcgtaggtgaggtcctcggggcattgcaggccgtgctgctcgagcgcccactcctggagatgtgggttggcttgcatgaatgaagcccagagctcgcgggccatgagggtctggagctcgtcgcgaaagaggcggaactgctggcccacggccatcttttctggggtgacgcagtagaaggtgagggggtcccgctcccagcgatcccagcgtaagcgcacggcgagatcgcgagcgagggcgaccagctcggggtccccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggtcgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatttcggcgcggcgggggcggagagcgaaaacgagggcgcgcagttgggagctgtccatggtgtcgcggagatccaggtccgggggcagggttctgaggttgacctcgtagaggcgggtgagggcgtgcttgagatgcagatggtacttgatctccacgggtgagttggtggtcgtgtccacgcattgcatgagcccgtagctgcgcggggccacgaccgtgccgcggtgcgcttttagaagcggtgtcgcggacgcgctcccggcggcagcggcggttccggccccgcgggcagtggcggtagaggcacgtcggcgtggcgctcgggcaggtcccggtgctgcgccctgagagcgctggcgtgcgcgacgacgcggcggttgacatcctggatctgccgcctttgcgtgaagaccacgggccccgtgactttgaacctgaaagacagttcaacagaatcaatctcggcgtcattgacggcggcctgacgcaggatctcttgcacgtcgcccgagttgtcctggtaggcgatctcggacatgaactgctcgatttcctcctcctggagatcgccgcggcccgcgcgctctacggtggcggcaaggtcattcgagatgcgacccatgagctgcgagaaggcgcccaggccgctctcgttccagacgcggctgtaaaccacgtccccgtcggcgtcgcgcgcgcgcatgaccacctgcgcgaggttgagctccacgtgccgcgtaaagacggcgtagttgcgcaggcgctggaagaggtagttgagggtggtggcgatgtgctcggtgacgaagaagtacataatccagcggcgcaggggcatttcgctgatgtcgccaatggcctccagcctttccatggcctcgtagaaatccacggcgaagttgaaaaactgggcgttgcgggccgagaccgtgagctcgtcttccaggagcctgatgagttcggcgatggtggcgcgcacctcgcgctcgaaatcccagggggcctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggtgggtccccgttgggcagcgatagggcgctgacaatgcatcttatcaattgcggtgtagggcacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttgaggcggcggatggtggcgaggaggaccaggtccttgggtcccgcttgctggatgcggagccgctcggccatgccccaggcctggccctgacaccggctcaggttcttgtagtagtcatgcatgagcctctcgatgtcatcactggcggaggcggagtcttccatgcgggtgaccccgacgcccctgaacggctgcacgagcgccaggtcggcgacgacgcgctcggcgaggatggcctgttgcacgcgggtgagggtgtcctggaagtcgtccatgtcgacgaagcggtggtaggcccctgtgttgatggtgtaagtgcagttggccataagcgaccagttgacggtctgcaggccgggttgcacgacctcggagtacctgagccgcgagaaggcgcgcgagtcgaagacatagtcgttgcaggtgcgcacgaggtactggtatccgactagaaagtgcggcggcggctggcggtagagcggccagcgctgggtggccggcgcgcccggggccaggtcctcaagcatgagtcggtggtagccgtagaggtagcgggacatccaggtgatgccggcggcggtggtggaggcgcgcgggaactcgcggacgcggttccagatgttgcgcaggggcaggaaatagtccatggtcggcacggtctggccggtgagacgcgcgcagtcattgatgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccaagcccgatagccgccaggatacggcggagagccctttttgtcggccgaggggagtcgctagacttgaaagcggccgaaaaccctgccgggtagtggctcgcgcccgtagtctggagaagcatcgccagggttgagtcgcggcagaacccggttcaaggacggccgcggcgagcgggacttggtcaccccgccgatttaaagacccacagccagccgacttctccagttacgggagcgagcccccttttttctttttgccagatgcatcccgtcctgcgccaaatgcgtcccacccccccggcgaccaccgcgaccgcggccgtagcaggcgccggcgctagccagccacagccacagacagagatggacttggaagagggcgaagggctggcgagactgggggcgccgtccccggagcgacatccccgcgtgcagctgcagaaggacgtgcgcccggcgtacgtgcctgcgcagaacctgttcagggaccgcagcggggaggagcccgaggagatgcgcgactgccggtttcgggcgggcagggagctgcgcgagggcctggaccgccagcgcgtgctgcgcgacgaggatttcgagccgaacgagcagacggggatcagccccgcgcgcgcgcacgtggcggcggccaacctggtgacagcctacgagcagacggtgaagcaggaacgcaactttcaaaagagtttcaacaaccacgtgcgcaccctgatcgcgcgcgaggaggtggccctgggcctgatgcacctgtgggacctggcggaggccattgtgcagaacccggacagcaagcctctgacggcacaactgttcctggtggtgcagcacagcagggacaacgaggcgttcagggaggcgctgctaaacatcgccgagcccgagggccgctggctgctggagctgatcaacatcttgcaaagcatcgtagtgcaggagcgcagcctgagcttggccgagaaggtggcggcgatcaactactcggtgctaagcctgggcaagttttacgcgcgcaagatttacaagacgccgtacgtgcccatagacaaggaggtgaaaatagacagcttttacatgcgcatggcgctcaaggtgctgacgctgagcgacgacctgggcgtgtaccgcaacgaccgcatccacaaggccgtgagcacgagccggcggcgcgagctgagcgaccgcgagctgatgctaagcctgcgccgggcgctggtaggtggcgccgccggcggcgaggagtcctacttcgacatgggggcggacctgcattggcagccgagccggcgcgccttggaggccgcctacggtccagaggacttggatgaggatgaggaagaggaggaggatgcacccgttgcggggtactgacgcctccgtgatgtgtttttagatgtcccagcagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactttcgaccattctggaggcggtggtcccctctcggaccaaccccacgcacgagaaggtgctggcgatcgtgaacgcgctggcggagaacaaggctattcgtcccgacgaggctgggctggtatacaacgccctgctggagcgcgtgggccgctacaacagcacgaacgtgcagtccaacctggaccggctggtgacggacgtgcgcgaggccgtggcgcagcgcgagcggttcaagaacgagggcctgggctcgctggtggcgctgaacgccttcctggcgacgcagccggcgaacgtgccgcgcgggcaggacgattataccaactttatcagcgcgctgcggctgatggtgaccgaggttccccagagcgaggtgtaccagtcgggcccggactactttttccagactagcagacagggcctgcagacggtgaacctgagccaggctttcaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggtcacctgctgacgctgtaccgcgaggccataggccaggcacaggtggacgagcagaccttccaggagatcactagtgtaagccgcgcgctgggtcagaacgacaccgacagtctgagggccaccctgaacttcttgctgaccaatagacagcagaagatcccggcgcagtatgcgctgtcggccgaggaggagcgcatcctgagatatgtgcagcagagcgtagggctgtttctgatgcaggagggggccacccccagcgccgcgctggacatgaccgcgcgcaacatggaacctagcatgtacgccgccaaccggccgtttatcaataagctgatggactacctgcaccgcgcggcgtccatgaactcggactactttaccaatgccattttgaacccgcactggctcccgccgccggggttctacacgggcgagtacgacatgcctgaccccaacgacgggtttttgtgggacgacgtggacagcgcggtgttctcaccgaccttgcaaaagcgccaggaggcggtgcgcacgcccgcgagcgagggcgcggtgggtcggagcccctttcctagcttagggagtttgcatagcttgccgggctcggtgaacagcggcagggtgagccggccgcgcttgctgggcgaggacgagtacctaaacgactcgctgctgcagccgccgcgggtcaagaacgccatggccaataacgggatagagagtctggtggacaaactgaaccgctggaagacctacgctcaggaccatagggagcctgcgcccgcgccgcggcgacagcgccacgaccggcagcggggcctggtgtgggacgacgaggactcggccgacgatagcagcgtgttggacttgggcgggagcggtggggtcaacccgttcgcgcatctgcagcccaaactggggcgacggatgttttgaatgcaaaataaaactcaccaaggccatagcgtgcgttctcttccttgttagagatgaggcgtgcggtggtgtcttcctctcctcctccctcgtacgagagcgtgatggcgcaggcgaccctggaggttccgtttgtgcctccgcggtatatggctcctacggagggcagaaacagcattcgttactcagagctggctccgctgtacgacaccactcgcgtgtacttggtggacaacaagtcggcggacatcgcttccctgaactaccaaaacgaccacagcaactttctgaccacggtggtgcaaaacaacgatttcacccccgccgaggctagcacgcagacgataaattttgacgagcggtcgcggtggggcggtgatctgaagaccattctgcacaccaacatgcccaatgtgaacgagtacatgtttaccagcaagtttaaggcgcgggtgatggtggctaggaaacacccacagggggtagaagcaacagatttaagcaaggatatcttagagtaccagtggtttgagtttaccctgcccgagggcaacttttccgagaccatgaccatagacctgatgaacaacgccatcttggaaaactacttgcaagtggggcggcaaaatggcgtgctggagagcgatatcggagtcaagtttgacagcaggaatttcaagctgggctgggaccccgtgaccaagctggtgatgccaggggtctacacctatgaggccttccacccggacgtggtgctgctgcctggctgcggggtggacttcaccgagagccgcctaagcaaccttctgggcattcgcaagaagcaacctttccaagagggcttcagaatcatgtatgaggatctcgaagggggcaacattcccgcacttctgaatgtgaccaagtacctggaaagcaagaagaagctagaggagaatgccgctaaggctaatggtcctgcaagaggagacagtagtgtctcaagagaggtggaaaaggcagctgaaaaagagcttgtcattgagcccatcaagcaagatgatagcaagagaagttacaacctcattgagggtacccatgacaccctgtaccgaagctggtacctgtcctatacctacggggaccccgagaagggggtgcagtcgtggacgctgctcaccaccccggacggtcactgcggcgcggagcaagtctactggtcgctgccggacctcatgcaagaccccgtcaccttccgctctacccagcaagtcagcaactaccccgtggtcggcgccgagctcatgcctttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctttccagtcgcaccttctaaaaaatgtctattctcatctcgcccagcaataacaccggctggggtcttactaggcccagcaccatgtacggaggagccaagaaacgctcccagcagcaccccgtccgcgtccgcggccactttcgcgctccctggggcgcatacaagcgcgggcggacttccaccgccgccgccgtgcgcaccaccgtcgacgacgtcatcgactcggtggtcgccgatgcgcgcaactatacccccgccccctccaccgtggacgcggtcattgacagcgtggtggccgacgcgcgcgactatgccagacgcaagagccggcggcgacggatcgccaggcgccaccggagcacgcccgccatgcgcgccgcccgggctctgctgcgccgcgccagacgcacgggccgccgggccatgatgcgagccgcgcgccgcgctgccactgcacccacccccgcaggcaggactcgcagacgagcggccgctgccgccgccgcggccatctctagcatgaccagacccaggcgcggaaacgtgtactgggtgcgcgactccgtcacgggcgtgcgcgtgcccgtgcgcactcgtcctcctcgtccctgatctaatgcttgtgtcctcccccgcaagcgacgatgtcaaagcgcaaaatcaaggaggagatgctccaggtcgtcgccccggagatttacggacccccggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgtgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggttaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcaaaccgtggacattcagatccccaccgacatggatgtcgacaaaaaaccctcgaccagcatcgaggtgcaaaccgacccctggctcccagcctccaccgctaccgccgccacggccaccgagcctcccaggaggcgaagatggggccctgccaaccggctgatgcccaactacgtgttgcatccttccatcatcccgacgccgggctaccgcggcacccggtactacgccagccgcaggcgcccagccagtaaacgccgccgccgcaccgccacccgccgccgtctggcccccgcccgcgtgcgccgcgtgaccacgcgccggggccgctcgctcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagtggcctgaaccgccgccggcggcgggccatgcgcaggcgcctgagtggcggctttctgcccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcactgggcagttgttaagggaaaagctcaaagaccagaacttccagcaaaaggtggtggacgggctggcctcgggcattaacggggtggtggacatcgcgaacccaggccgtgcagcgcgagataaacaaccgcctggacccgcggccgcccacggtggtggagatggaagatgcaactcctccgccgcccaagggcgagaagcgaccgcggcccgacgcggaggagacgatcctgcaggtggacgagccgccctcgtacgaggaggccgtaaaggccggcatgcccaccacgcgcatcatcgcgccactggccacgggtgtaatgaaacccgccacccttgacctgcctccaccacccacgcccgctccaccgaaggcagctccggtagtgcagccccctccggtggcgaccgccgtgcgccgcgtccccgcccgccgccaggcccaaaactggcaaagcacgctgcacagtattgtgggcctgggagtgaaaagtctgaagcgccgccgatgctattgaaagagaggaaggaagacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccaacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgttctggaccgcggccccagcttcaaaccctactcgggcacggcttacaacagcctggcccccaagggcgcccccaattccagtcagtgggatgctcaagaaaaaaatggacaaggaggaaatgacatggttaccaaaactcacacatttggcgtggctgctatgggaggaacaaatattacaaaccagggtttgttaattggaactgaagaaacagccgataatcctccaaaggaaatctttgcagacaaattattccagccagaacctcaagtaggagaggaaaactggcaagacagcaatgcattctatggaggcagggctcttaagaaggaaactaaaatgaaaccatgctatggatcttatgctagaccaacaaacacaagtggcggacaggctaagcttaaaactggtgacaatatcgatcctaccaaggatttcgacatagatcttgctttcttcgatactcctggcggaaatcctccagcaggtggtagtggaacggaagaatacaaagcagatattgttatgtacactgaaaatgtcaaccttgaaacacctgacactcatgtggtgtacaaaccagccaaagaggatgaaagttctcaggccaacttggttcagcagtccatgcccaacagacccaactacattggcttcagagacaattttgtggggctcatgtattacaacagcactggcaacatgggagtgctggctggtcaggcctctcagttgaatgctgtggtggacttgcaagacagaaacacagagctgtcttaccagctcttgctagattctctgggtgacagaaccagatactttagcatgtggaactctgcggtggacagctatgatccagatgtcagaatcattgaaaatcacggtgtggaagatgagcttccaaactattgctttccattggatggctctggtaccaatgctgcctaccaaggtgtaaaggttcaagatggtgaagacggggataaagaaactgaatgggaaaaagataccaaagtcgcagatcgtaaccaactgtgcaagggtaacatcttcgccatggagatcaacctccaggccaacctgtggaagagttttctgtactcgaacgtggccctgtacctgcccgactcctacaagtacacgccggccaacatcacgctgcccgccaacaccaacacctacgagtacatgaacggccgcgtggtagccccctcgctggtggacgcatacgtcaacatcggtgcgcgctggtcgctggaccccatggacaacgtcaaccccttcaaccaccaccgcaacgcgggcctgcgctaccgctccatgcttctcggcaacggccgctacgtgcccttccacatccaagtgccccaaaagttctttgccattaagaacctgctcctgctccccggctcctacacctacgagtggaacttccgcaaggatgtcaacatgatcctgcagagttccctcggaaacgacctgcgcgtcgacggcgcctccgtgcgcttcgacagcgtcaacctctacgctaccttcttccccatggcgcacaacaccgcctccaccctggaagccatgctgcgcaacgacaccaacgaccagtcctttaacgactacctctcggccgccaacatgctctaccccataccggccaaggccaccaacgtgcccatctccatcccctcgcgcaactgggctgccttccgcggctggagtttcacccggctcaagaccaaggaaactccttcccttggctcgggtttcgacccctactttgtctactcgggctccatcccctacctcgacgggaccttctacctcaaccacaccttcaaaaaggtgtccattatgttcgactcctcggtcagctggcccggcaacgaccggctgctcacgccgaatgagttcgagatcaagcgcagcgtcgacggggagggctacaacgtggcccaatgcaacataaccaaggactggttcctcgtccagatgctctcccactacaacatcggctaccagggcttccacgtgcccgagggctacaaggaccgcatgtactcctttttccgcaacttccagcccatgagcaggcaggtggtggatgagatcaactacaaggactacaaggccgtcaccctgcccttccagcacaacaactctggcttcaccggctacctcgcacccaccatgcgtcaggggcagccttaccccgccaacttcccttacccgctcatcggctccaccgcagtcccctccgtcacccagaaaaagttcctctgcgacagggtcatgtggcgcatccccttctccagcaacttcatgtccatgggtgccctcaccgacctgggtcagaacatgctctatgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacacccttctccgccggcaacgccaccacctaagcatgagcggttccagcgaacgagaactcgcggccatcgtgcgcgacctgggctgcgggccctactttttgggcacccacgacaagcgcttcccgggcttcctagccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgaagcgccctggcctcctcgcccgaccgctgtctcagcctcgaacagtccacccagaccgtgcaggggcccgactccgccgcctgcggacttttttgttgcatgttcttgcatgcgttcgtgcactggcccgaccgacccatggacggaaaccccaccatgaacttgctgacgggggtgcccaacggcatgctacaatcgccacaggtgctgcccaccctccggcgcaaccaggaggagctctaccgcttcctcgcgcgccactccccttacttccgatcccaccgcgccgccatcgaacacgccaccgcttttgacaaaatgaaacaactgcgtgtatctcaataaacagcactttttattttacatgcactggagtatatgcaagttatttaaaagtcgaaggggttctcgcgctcgtcgttgtgcgccgcgctggggagggccacgttgcggtactggtacttggaaagccacttgaactcggggatcaccagtttgggcactggggtctcggggaaggtctcgctccacatgcgccggctcatctgcagggcgcccagcatgtcagggccggagatcttgaaatcacagttggggccggtgctctgcgcgcgcgagttgcggtacacggggttgcagcactggaacaccatcagactggggtacttcacactggcaagcacgctcttgtcgctaatctgatccttgtccaggtcctcggcgttgctcaggccgaacggggtcatcttgcacagctggcggcccaggaagggcacgctctgaggcttgtggttacactcgcagtgcacgggcatcagcatcatccccgcgccgcgctgcatattcgggtagagggccttgacgaaggccgcgatctgcttgaaagcttgctgggccttggccccctcgctgaagaacagaccgcagctcttcccgctgaactggttattcccgcacccggcatcatgcacgcagcagcgcgcgtcatggctggtcagttgcaccacgctccgtccccagcggttctgggtcaccttagccttgctgggctgctccttcagcgcgcgctgtccgttctcgctggtcacatccatctccaccacgtggtccttgtgaatcatcaccgttccatgcagacacttgagctgaccttccacctcggtgcagccgtgatcccacaggacgcagccggtgcactcccaattcttgtgcgcgatcccgctgtggctgaaaatgtaaccttgcaacaggcgacccataatggtgctaaatgatttctgggtggtgaatgtcagttgcatcccgcgggcctcctcgttcatccaggtctggcacatcttctggaagatctcggtctgctccggcatgagcttgtaagcatcgcgcaagccgctgtcgacgcggtagcgttccatcagcacgttcatggtatccatgcccttctcccatgacgagaccagaggcagactcagggggttgcgcacgttcaggacaccaggggtcgcgggctcgacgatgcgttttccgtccttgccttccttcaacagaaccggaggctggctgaatcccactcccacgatcacggcgtcttcctggggcatctcttcgtcggggtctaccttggtcacatgcttggtctttctggcttgcttcttttttggagggctgtccacggggaccacgtcctcctcggaagacccggagcccacccgctgatactttcggcgcttggtgggcagaggaggtggcggcggcgaggggctcctctcctgctccggcggatagcgcgccgacccgtggccccggggcggagtggcctctcgctccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctaaaacccccacaggatgaacaggagcacgagcaagacgcaggccaggaggagaccgacgctgggctcgagcatggctacctgggaggagaggaggatgtgctgctaaaacacctgcagcgccagtccctcatcctccgggacgccctggccgaccggagcgaaacccccctcagcgtcgaggagctgtgtcgggcctacgagctcaacctcttctcgccgcgcgtgccccccaaacgccagcccaacggcacctgcgagcccaacccgcgtctcaacttctatcccgtctttgcggtccccgaggcccttgccacctatcacatctttttcaagaaccaaaagatccccatctcctgtcgcgccaatcgcactcgcgccgacgcgctcctcgctctggggcccggcgcgcgcatacctgatatcgcttccctggaagaggtgcccaagatcttcgaagggctcggtcgggacgagacgcgcgcggcaaacgctctgaaagaaacagcagaggaagagggttacactagcgccctggtagagttggaaggcgacaacgccaggctggccgtgcttaagcgcagcgtcgagctcacccatttcgcctaccccgccgtcaacctcccgcccaaggtcatgcgtcgcatcatggatcagctcatcatgccccacatcgaggcccttgatgaaagtcaggaacagcgccccgagaacgcccagcccgtggtcagcgacgagatgctcgcgcgctggctcgggacccgcgacccccaggccctggagcagcggcgcaagctcatgctggccgtggtcctggtcacccttgagctcgaatgcatgcgccgcttttttaccgaccccgagaccctgcgcaaggtcgaggagaccctgcactacactttcagacacggtttcgtcaggcaggcctgcaagatctccaacgtggagctgaccaacctggtctcctgcctggggatcctacacgagaaccgcttgggacagaccgtgctccactctaccctgaagggcgaggcgcggcgggactacatccgcgactgcgtctttctctttctctgccacacatggcaagcggccatgggcgtgtggcagcagtgtctcgaggacgagaacctgaaggagctggacaagcttcttgctagaaaccttaaaaagctgtggacgggcttcgacgagcgcaccgtcgcctcggacctggccgagatcgtcttccccgagcgcctgaggcagacgctgaaaggagggctgcccgacttcatgagccagagcatgttgcaaaactaccgcactttcattctcgagcgatctgggatgctgcccgccacctgcaacgccttcccctccgactttgtcccgctgagctaccgcgagtgtcccccgccgctgtggagccactgctacctcttgcagctggccaactacattgcccaccactcggatgtgatcgaggacgtgagcggcgaggggctgctcgagtgccactgtcgctgcaacctatgctccccgcaccgctccctggtctgcaacccccagctactgagcgagacccaggtcatcggtacctttgagctgcaaggtccgcaggagtccaccgctccgctgaaactcacgccggggttgtggacttccgcgtacctgcgcaaatttgtacccgaggactactacgcccatgagataaagttcttcgaggaccaatcgcgtccgcagcacgcggatctcacggcctgcgtcatcacccagggcgcgatcctcgcccaattgcacgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagacttggaagaggtggaagaggagcaggcaacagagcagcccgtcgccgcaccatccgcgccggcagcccctccggtcacggatacaacctccgcagctccggccaagcctcctcgtagatgggatcgagtgaagggtgacggtaagcacgagcgacagggctaccgatcatggagggcccacaaagccgcgatcatcgcctgcttgcaagactgcggggggaacatcgctttcgcccgccgctacctgctcttccaccgcggggtgaacatcccccgcaacgtgttgcattactaccgtcaccttcacagctaagaaaaagcaagtcaaaggagtcgccggaggaggaggcctgaggatcgcggcgaacgagcccttgaccaccagggagctgaggaaccggatcttccccactctttatgccatttttcagcaaagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctgactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggcttagtgccggcccctcgatgatctcacgggtcaacggggtccgtaaccatcgaaaccagatattgttgcagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagctcaacgacgagttggtgagctcttcaatcggtctgcgaccggacggagtgttccaactagccggagccgggagatcgtccttcactcccaaccaggcctacctgaccttgcagagcagctcttcggagcctcgctcgggaggcatcggaaccctccagttcgtggaggagtttgtgccctcggtctacttcaaccccttctcgggctcgccaggcctctacccggacgagtttataccgaacttcgacgcagtgagagaagcggtggacggctacgactgaatgtcctatggtgactcggctgagctcgctcggttgaggcatctggaccactgccgccgcctgcgctgctttgcccgggagagctacggcctcatctactttgagctgcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatttgtcctaccccgaagttgcatgagaatttttgttgtactctttgtggtgagtttaataaaagctaaactcttgcaatactctggaccttgtcgtcatcaactcaacgagaccgtctacctcaccaaccagactgaggtaaaacttacctgcagaccacacaagacctatatcatctggttcttcgagaacacctcatttgcagtctccaacactcactgcaacgacggtgttgaacttcccaacaacctttccagtggactgagttacaatacacgtagagctaagctcatcctctacaatccttttgtagagggaacctaccagtgccagagcggaccttgcttccacagttttactttggtgaacgttaccggcagcagcacagccgctccagaaactaaccttccttctgatactatcaaaccttgtttcggaggtgagctaaggcttcccccttctcaggagggggttagcccatacgaagtggtcgggtatttgattttaggggtggtcctgggtgggtgcatagcggtgctagctcagctgccttgctgggtggaaatcaaaatctttatatgctgggtaagacattgtggggaggaactatgaaggggctcttgctgattatcctttccctggtggggggtgtgctgtcatgccacgaacagccacgatgtaacatcaccacaggcaatgagaggaacgactgctctgtagttatcaaatgcgagcaccattgtcctctcaacattacattcaaaaataagaccatgggaaatgtatgggtgggattctggcaaccaggagatgagcagaactacacggtcactgtccatggtagcaatggcaatcacactttcggtttcaaattcatttttgaagtcatgtgtgatatcacactacatgtggctagacttcatggcttgtggccccctaccaaggataacatggtgggtttttctttggcttttgtgatcatggcctgcttgatgtcaggtctgctggtaggggctctagtgtggtttctgaaacgcaagcccaggtatggaaatgaagagaaggaaaaattgctataaattctttttctttttcgcagaaccatgaatacagtgatccgtatcgtgctgctctctcttcttgtagcttttagtcaggcaggatttcatactatcaatgctacatggtgggctaatataactttagtgggacccccagacacaccagtcacttggtatgatactcaaggattgtggttttgcaatggcagtagagttaagaatcctcaaatcagacatacatgtaatgatcaaaaccttactttgatccatgtgaacaaaacttatgaaagaacatacatgggttataatagacaagggactaaaaaagaagactacaaagttgtagttataccacctcctcctgctactgtaaaaccacagccagagccagagtatgtgtttgtttatatgggagagaacaaaactctagaaggtcctccgggaactccagtcacatggtttaatcaggatggaaagaaattttgtgaaggagaaaaagttcttcatccagaatttaaccacacctgtgacaaacaaaaccttatactactgtttgtgaattttacacatgatggagcttaccttgggtacaatcatcaaggaacccagagaacacactatgaagttacagtattagatctttttccagattctggccaaatgaaaattgaaaatcatagtgaggaaacagagcaaaaaaatgatgaacatcataactggcagaaacagggtgggcaaaaacagggtgggcaaaaaacaaatcaaacaaaagttaatgacaggagaaaaacagcgcaaaaaagaccatcaaagctaaagccggcaactattgaggcaatgctggttacagtgactgccgggtctaacttaactttggttggacctaaagcagaaggaaaagttacttggtttgatggagatttaaaaagaccatgtgagcctaattacagactaagacacgaatgtaataatcaaaacttaactctgattaatgtaactaaagattatgagggaacttactatggtacaaatgacaaagatgagggcaaaaggtacagagtgaaagtaaatactacaaattctcaatctgtgaaaattcagccatataccagacaaactactcctgatcaagagcacaaatttgaattacagttcgaaactaatggaaattatgattcaaaaattccctcaaccactgtggcaatcgtggtgggtgtgattgcgggcttcataactctgatcattgtcttcatatgctacatctgctgccgcaagcgtcccagggcatacaatcatatggtagacccactactcagcttctcttactaagactcagtcactttcatttcagaaccatgaaggctttcacagcttgcgttctgattagcctagtcacacttagtgtagctattaaaaatcaatatcatgttcataatgttaccagagatggatatatcacattaaatgtaacaattgataatactacctggacaagatatcatttaaataagtggcatcaaatttgtacgtggtcagacccatcatacaaatgtcacagcaatggcagcattaccattcatgctttcaatattacttctggccagtacaaagctgaaagttttactaactggtttagatattacggtaatcataaacatgaaattcatatttttaacataactgtaattgagcatcctacaacaaaagcacccaccactgctaatacagctacatcaattaaatcaacaaccacacagcctactactagggagacaactcaacctaccaccacagtcagtacaactactgagaccactactcaaactacacagctagacacaacagtgcagaatagcactgtgttggttaggtatctgttgagggaggaaagtactactgaacagacagaggctacctcaagtgcctttagcagcactgcaaatttaacttcgcttgcttggactaatgaaaccggagtatcattgatgaatcatcagcctttctcaggtttggatattcaaattacttttctggttgtttgtgggatctttattcttgtggttcttctgtactttgtctgctgcaaagccagagagaaatctaggaggcccatctacaggccagtaatcggggaacctcagccactccaagtggaagggggtctaaggaatcttcttttctctttttcagtatggtgatcagccatgattcctaggttcttcctatttaacatcctcttctgtctcttcaacatctgcgctgcctttgcagccgtctcgcacgcctcgcccgactgtctcgggcccttcccaacctacctcctctttgccctgctcacctgcacctgcgtctgcagcattgtctgcctggtcatcaccttcctgcagcttatcgactggtgctgtgcgcgctacaattatctccatcacagtcccgaatacagggacaagaacgtagccagaatcttaaggctcatctgaccatgcagactctgctcatgctgctatccctcctatcccctgccctagccacttatgctgattactctaaatgcaaattcgcagacatatggaatttcttagattgctatcaggaaaaaattgatatgccctcctattacttggtgattgtgggaatagtcatggtctgctcctgcactttctttgccatcatgatttacccctgttttgatctcggctggaactctgttgaagcattcacatacacactagaaagcagttcactagcctccacgccaccacccacaccgcctcctcgcagaaatcagttccccctgatacagtacttagaagagccccctccccgacccccttccactgttagctactttcacataaccggcggcgatgactgaccaccacctggacctcgagatggacggccaggcctccgagcagcgcatcctgcaactgcgcgtccgtcagcagcaggagcgggccgccaaggagctccttgatgccatcaacatccaccagtgcaagaagggcatcttctgcctggtcaaacaggcaaagatcacctacgagctcgtgtccaacggcaaacagcatcgccttacctatgagatgccccagcagaagcagaagttcacctgcatggtgggcgtcaaccccatagtcatcacccagcagtcgggcgagaccaacggctgcatccactgctcctgcgaaagccccgagtgcatctactcccttctcaagaccctttgcggactccgcgacctcctccccatgaactgatgttgattaaaagcccagaaaccaatcagacccttcctcatttccccatcccaatactcataagaataaatcattggaattaatcattcaataaagatcacttacttgaaatctgaaagtatgtctctggtgtagttgctcagcaacacctcggtaccctcctcccagctctggtactccagtccccggcgggcggcgaacttcctccacaccttgaaagggatgtcaaattcctggtccacaattttcattgtcttccctctcagatgtcaaagaggctccgggtggaagatgacttcaaccccgtctacccctatggctacgcgcggaatcagaatatccccttcctcactcccccctttgtctcctccgatggattcaaaaacttcccccctggggtactgtcactcaaactggctgatccaatcaccattaccaatggggatgtatccctcaaggtgggaggtggtctcactttgcaagatggaagcctaactgtaaaccctaaggctccactgcaagttaatactgataaaaaacttgagcttgcatatgataatccatttgaaagtagtgctaataaacttagtttaaaagtaggacatggattaaaagtattagatgaaaaaagtgctgcggggttaaaagatttaattggcaaacttgtggttttaacaggaaaaggaataggcactgaaaatttagaaaatacagatggtagcagcagaggaattggtataaatgtaagagcaagagaagggttgacatttgacaatgatggatacttggtagcatggaacccaaagtatgacacgcgcacactttggacaacaccagacacatctccaaactgcacaattgctcaagataaggactctaaactcactttggtacttacaaagtgtggaagtcaaatattagctaatgtgtctttgattgtggtcgcaggaaagtaccacatcataaataataagacaaatccaaaaataaaaagttttactattaaactgctatttaataagaacggagtgcttttagacaactcaaatcttggaaaagcttattggaactttagaagtggaaattccaatgtttcgacagcttatgaaaaagcaattggttttatgcctaatttggtagcgtatccaaaacccagtaattctaaaaaatatgcaagagacatagtttatggaactatatatcttggtggaaaacctgatcagccagcagtcattaaaactacctttaaccaagaaactggatgtgaatactctatcacatttaactttagttggtccaaaacctatgaaaatgttgaatttgaaaccacctcttttaccttctcctatattgcccaagaatgaaagaccaataaacgtgtttttcatttgaaattttcatgtatctttattgatttttacaccagcacgagtagacagtctcccaccaccagcccattttacagtgtacacggttctctcagcacgggtagccttaaatagggaaatattctcattagtgcgggaattggacttggggtctataatccacacagtttcctggcgagccaaacgggggtcggtgattgaaataaagccgtcctctgaaaagtcatccaagcgggcctcacagtccaaggtcacagtctggtggaacgagaagaacgcacagattcatactcggaaaacaggatgggtctgtgcctctccatcagcgccctcagcagtctctgccgccggggctcggtgcggctgctgcaaatgggatcgggatcacaagtctctctgactatgatcccaacagccttcagcatcagtctcctggtgcgacgggcacagcaccgcatcctgatctctgccatgttctcacagtaagtgcagcacataatcaccatgttattcagcagcccataattcagggcgctccagccaaagctcatgttgggaatgatggaacccacgtgaccatcgtaccagatgcgacagtatatcagatgcctgcccctcatgaacacactgcccatgtacatgatctctttgggcatgtttctgtttacaatctggcggtaccaggggaagcgctggttgaacatgcacccgtaaatgactctcctgaaccacacggccagcagggtgcctcccgcccgacactgcagggagccaggggatgaacagtggcaatgcaggatccagcgctcgtacccgctcaccatttgagctcttaccaagtccagggtagcggggcacaggcacactgacatacatctttttaaaatttttatttcctctgtggtgaggatcatatcccaggggactggaaactcttggagcagggtaaagccagcagcacatggtaatccacggacagaacttacattatgataatctgcatgatcacaatcgggcaacaggggatgttgttcagtcagtgaagccctggtttcctcatcagatcgtggtaaacgggccctgcgatatggatgatggcggagcgagctggattgaatctcggtttgcattgtagtggattctcttgcgtaccttgtcgtacttctgccagcagaaatgggcccttgaacagcatatacccctcctacggccgtcctttcgctgctgccgctcagtcatccaactaaagtacatccattctcgaagattctggagaagttcctctgcatctgataaaataaaaaacccgtccatgcgaattcccctcatcacatcagccaggactctgtaggccatccccatccagttaatgctgccttgtctatcattcagagggggcggtggcaggactggaagaaccatttttattccaaacggtctcgaaggacgataaagtgcaagtcacgcaggtgacagcgttcccctccgctgtgctggtggaaacagacagccaggtcaaaacccactctattttcaaggtgctcgaccgtggcttcgagcagtggctctacgcgcacatccagcataagaatcacattaaaggctggccctccatcgatttcatcaatcatcaggttacattcctgcaccatccccaggtaattctcatttttccagccttggattatctctacaaattgttggtgtaagtccactccgcacatgtggaaaagctcccacagtgccccctccactttcataatcaggcagaccttcataatagaaacagatcctgctgctccaccacctgcagcgtgttcaaaacaacaagattcaataaggttctgccctccgccctgagctcgcgcctcaatgtcagctgcaaaaagtcacttaagtcctgggccactacagctgacaattcagagccagggctaagcgtgggactggcaagcgtaagggaaaactttaatgctccaaagctagcacccaaaaactgcatgctggaataagctctctttgtgtctccggtgatgccttccaaaatgtgagtgataaagcgtggtagtttttctttaatcatttgcgtaatagaaaagtcctctaaataagtcactaggaccccagggaccacaatgtggtagcttacaccgcgtcgctgaagcatggttagtagagatgagagtctgaaaaacagaaagcatgcactaaactaaggtggctattttcactgaaggaaaaatcactctctccagcagcagggtacccactgggtggcccttgcggacatacaaaaatcggtccgtgtgattaaaaagcagcacagtaagttcctgtcttcttccggcaaaaatcacatcagactgggttagtatgtccctggcatggtagtcattcaaggccataaatctgccctgatatccagtaggaaccagcacactcacttttaggtgaagcaataccaccccatgcggaggaatgtggaaagattcagggcaaaaaaattatatctattgctagccccttcctggacgggagcaatccctccaggactatctataaaagcatacagagattcagccatagcttagcccgcttaccagtagacagaaagcacagcagtacaagcgccaacagcagcaactgactacccactgacccagctccctatttaaaggcaccttacactgacgtaatgaccaaaggtctaaaaaccccgccaaaaaaaacacacacgccctgggtgtttttcacaaaaacacttccgcgttctcacttcctcgtatcgattttgtgactcaacttccgggttcccacgttacgtcacttctgcccttacatgtaacttggccgtatggcgccatcttgcccacgtccaaaatggctttcatgaccggccacgcctccgcgccggccgttagccgtgcgtcgtgacgttatttgcatcaccgcttctcgtccaatcagcgttggctccgccccaaaaccgttaaaattcaaaagctcatttgcatattaacttttgtttactttgtggggtatattattagatag

SEQ ID NO: 2 - DNA, 22 bp, artificial
  ET upstream primer
tgtgggcggacaaaatagttgg

SEQ ID NO: 3 - DNA, 30 bp, artificial
  ET downstream primer
tgtgggcggacaataaagtcttaaactgaa